(19) United States
(12) Patent Application Publication
(10) Pub. No.: US 2025/0268765 A1
(43) Pub. Date: Aug. 28, 2025

(54) SANITARY ARTICLE WITH REMOVABLE LATERAL AND/OR END PORTIONS

(71) Applicant: c/o EASYDAY HEALTH PRODUCTS INC., Blainville (CA)

(72) Inventors: Cristian STEFU, Blainville (CA); Ioana Mihaela HIDISAN, Blainville (CA); Regent RACINE, Blainville (CA)

(21) Appl. No.: 19/205,611

(22) Filed: May 12, 2025

Related U.S. Application Data
(63) Continuation-in-part of application No. 17/419,015, filed on Jun. 28, 2021, now Pat. No. 12,295,818.

(51) Int. Cl. A61F 13/474; A61F 13/15; A61F 13/47; A61F 13/56; A61F 13/84

(52) U.S. Cl. CPC A61F 13/474; A61F 13/5616; A61F 2013/15878; A61F 2013/4708; A61F 2013/8497

(57) ABSTRACT

A sanitary article comprises a main body comprising a top and bottom sides and an absorbent core therebetween surrounded and bounded by a core border formed in the top and bottom sides. The top side defines a body contacting surface within the core border. The bottom side defines an undergarment contacting surface within the core border. A main body contour extends beyond the core border and is surrounded and bounded by a sealed main body perimeter defining opposite main body longitudinal ends and opposite main body lateral sides. Each main body lateral side having front and rear portions thereof and a middle portion therebetween. A respective tear line is defined between the inner lateral part of each one of the pair of wings and the middle portion. When a sufficient tearing force is applied to the tear line thereby a wing is fully removed from the main body without physical integrity disturbance of the sealed perimeter at each of the middle portions.

### CROSS-REFERENCE TO RELATED APPLICATIONS

The present application is a continuation-in-parts of U.S. patent application Ser. No. 17/419,015 which entered the national phase in the United States on Jun. 28, 2021 as a national stage entry of International Patent Application Number PCT/CA2019/000174 filed on Dec. 30, 2019 claiming priority on U.S. Provisional Patent Application Ser. No. 62/786,054 filed on Dec. 28, 2018 all of which are incorporated herein by reference in their entirety.

### TECHNICAL FIELD

The present disclosure generally relates to sanitary articles are as incontinence or human discharge protectors and are also in used feminine hygiene such as sanitary napkins or pads or incontinence napkins. More particularly but not exclusively the present disclosure relates to sanitary article with removable lateral and/or end portions.

### BACKGROUND

Sanitary articles are usually disposable absorbent articles designed to be worn externally of the body by a user and to receive fluids discharged from the body. Sanitary articles are used in feminine hygiene and they include sanitary napkins or pads. Sanitary articles also include incontinence napkins. Sanitary articles include panty liners, incontinence protectors and diapers. A sanitary napkin or pad is an absorbent sanitary article worn by a woman while menstruating, recovering from vaginal surgery, for lochia, after an abortion, or in any other situation where it is necessary to absorb a flow of blood from the vagina. These articles may also be used for incontinence. Of course, incontinence issues can also be managed by other sanitary articles such as incontinence napkins or pads. A sanitary article prevents soiling of the wearers clothing by such discharges. Sanitary articles include a body contacting side and a garment contacting side. Many sanitary articles have lateral portions such as wings, others do not have any laterally extending portions and users must choose between these available versions

### Objects

An object of the present disclosure is to provide a sanitary article.

An object of the present disclosure is to provide a sanitary article with removable lateral and/or end portions.

### SUMMARY

In accordance with an aspect of the present disclosure, there is provided sanitary article comprising: a longitudinal main body comprising a top body contacting surface, an opposite bottom undergarment contacting surface, and an absorbent core therebetween, the top body contacting surface and the bottom opposite outer surface being laminated to define laminated top and bottom surfaces formed about the absorbent core thereby defining an absorbent core sealed border line, the longitudinal main body is delimited and bounded by a perimeter defining sealed opposite ends and sealed opposite lateral margins, the sealed opposite ends define a main body length therebetween, the sealed opposite lateral margins define a main body width thereof and run along the length of the main body main, the lateral margins define respective lateral margin lengths thereof, the sealed opposite margins being respectively spaced apart from the absorbent core sealed border line by the laminated top and bottom surfaces therebetween; and two lateral wings respectively laterally extending from the main body beyond a respective one of the sealed opposite lateral margins and defining respective outer free edges thereof, each one of the two lateral wings having an undergarment contacting portion having an adhesive for adhesively contacting the undergarment of a wearer, wherein when adhesively contacting the undergarment of the wearer, each one of the two lateral wings extends beyond a crotch portion of the undergarment and is flipped thereunder and adhered to an outer surface of the undergarment opposite a body contacting surface of the undergarment, each one of the two lateral wings comprising a respective tear line formed thereon and being positioned adjacent of and spaced apart from a respective one of sealed opposite lateral margins running along the respective lateral margin length thereof and extending from one part of the free edge to an opposite part of the free edge, the respective tear line of each one of the two lateral wings defining an outer lateral wing portion and an inner lateral wing portion, the inner lateral wing portion being positioned between the tear line and the adjacent sealed lateral margin, wherein the respective tear line of each one of the pair of lateral wings provides for both maintaining the outer lateral wing portion attached to inner lateral wing portion when each of the lateral wings is adhesively contacting the undergarment and for detaching the outer lateral wing portion from the inner lateral wing portion when a sufficient tearing force is applied thereto thereby providing for fully removing each of the lateral wings from the longitudinal main body without physical integrity disturbance of each of the sealed opposite lateral margins.

In an embodiment, each of the sealed opposite lateral margins defining front and rear portions thereof and a middle portion therebetween; each of the two lateral wings respectively laterally extending from the middle portion of a respective one of the sealed opposite lateral margins; each of the outer free edges of each of the two lateral wings defining front and rear edge sections thereof interconnected by a middle edge section therebetween, the front and rear edge sections laterally extending away from the middle portion of a respective one of the sealed opposite lateral margins and the middle edge section being laterally spaced apart from the middle portion of a respective one of the sealed opposite lateral margins; each of the two lateral wings defining with the main body a respective front corner and a respective rear corner, the front corner formed at a junction between the front edge section of a respective one of the two lateral wings and the front portion of a respective one of the sealed opposite lateral margins, the rear corner formed at a junction between the rear edge section a respective one of the two lateral wings and the rear portion of a respective one of the sealed opposite lateral margins, and the tear line extending from the front corner to the rear corner along a length of the middle portion of a respective one of the sealed opposite lateral margins.

In an embodiment, the tear line comprises cut lines having relatively longer and short lengths for modulating tensile strength along the tear line.

In an embodiment, the tear line provides for an interdigitating pattern between outer lateral contour portion of the main body and the inner lateral portion of an adjacent one of the pair of wings along the tear line.

In an embodiment, the tear line of at least one of the lateral wings is a first tear line defining a first lateral margin length thereof and wherein the at least one lateral wing comprises a second tear line formed thereon and being positioned adjacent of and spaced apart from the first tear line running along the first lateral margin length thereof, wherein the second tear line separates the outer portion of the lateral wing into a first outer portion and a second outer portion, wherein the second outer portion is positioned between the first and second tear lines, wherein the second tear line provides for detaching the first outer portion from the second outer portion and wherein the first tear line provides for detaching the second outer portion from the inner portion.

In an embodiment, the first and second outer portions comprise respective indicia.

In an embodiment, the opposite bottom undergarment contacting surface comprises an adhesive material.

In accordance with an aspect of the present disclosure, there is provided a sanitary article comprising: a main body comprising a top side, an opposite bottom side and an absorbent core therebetween surrounded and bounded by a core border formed in the top and bottom sides, the top side defining a body contacting surface within the core border, the bottom side defining an undergarment contacting surface within the core border, a main body contour extending beyond the core border and surrounded and bounded by a sealed main body perimeter defining opposite main body longitudinal ends and opposite main body lateral sides, each of the main body lateral sides having front and rear portions thereof and a middle portion therebetween; and a pair of wings, each of the pair of wings laterally extending from the middle portion of a respective one of the main body lateral sides, each of the pair of wings defining an inner lateral part being contiguous with the middle portion of a respective one of the main body lateral sides and an outer free edge defining front and rear edge sections thereof interconnected by a middle edge section therebetween, the front and rear edge sections laterally extending away from the middle portion of a respective one of the main body lateral sides and the middle edge section being laterally spaced apart from the middle portion of a respective one of the lateral sides, each of the pair of wings having a respective undergarment contacting portion having an adhesive for adhesively contacting the undergarment of a wearer, wherein when adhesively contacting the undergarment of the wearer, each of the pair of wings extends beyond a crotch portion of the undergarment and is flipped thereunder and adhered to an outer surface of the undergarment opposite a body contacting surface of the undergarment, each of the pair of wings defining with the main body a respective front corner and a respective rear corner, the front corner formed at a junction between the front edge section of a respective one of the pair of wings and the front lateral side portion of a respective one of the main body lateral sides, the rear corner formed at a junction between the rear edge section a respective one of the pair of wings and the rear lateral side portion of a respective one of the main body lateral sides, and a respective tear line defined between the inner lateral part of each one of the pair of wings and the middle portion of a respective one of the main body lateral sides in contiguity therewith and extending from the front corner to the rear corner along a length of the inner lateral part of each one of the pair of wings and the middle portion of a respective one of the main body lateral sides in contiguity therewith; wherein each of the respective tear lines provides for both maintaining each of the pair of wings attached to the middle portion of a respective one of the main body lateral sides when each of the pair of wings is adhesively contacting the outer surface of the undergarment and for detaching each of the pair of wings from the middle portion of a respective one of the main body lateral sides in contiguity therewith when a sufficient tearing force is applied thereto thereby providing for fully removing each of the pair of wings from the main body without physical integrity disturbance of the sealed perimeter at each of the middle portions.

In an embodiment, the inner lateral portion of each of the pair of wings and the middle portion of a respective one of the main body lateral sides in contiguity therewith comprise compressed material. In an embodiment, the compressed material is heat sealed.

In an embodiment, the tear line comprises cut lines having relatively longer and short lengths for modulating tensile strength along the tear line.

In an embodiment, the tear line provides for an interdigitating pattern between the inner lateral portion of each of the pair of wings and the middle portion of a respective one of the main body lateral sides in contiguity therewith along the tear line.

In an embodiment, the undergarment contacting surface of the main body comprises an adhesive material.

In an embodiment, each of the pair of wings comprises inner tear lines for separating each of the pair of wings into sub-parts thereof.

In accordance with an aspect of the present disclosure, there is provided a sanitary article comprising: a main body defining opposite longitudinal ends thereof, opposite lateral sides thereof and comprising a top side, an opposite bottom side and an absorbent core therebetween bounded by a core border formed in the top and bottom sides, the top side defining a body contacting surface within the core border, the bottom side defining a garment contacting surface within the core border, an inner contour extending beyond the core border and bounded by a sealed inner contour border, an outer contour extending beyond the inner contour border and being bounded by a perimeter, the outer contour defining lateral outer contour portions at respective ones of the opposite lateral sides and a pair of wings, each of the pair of wings comprising an inner lateral portion extending beyond a respective one of the lateral outer contour portions and comprising an inner lateral portion being contiguous with the respective one of the lateral outer contour portions and forming a respective longitudinal junction therewith comprising tear line along a length thereof, the longitudinal junction comprising compressed material providing for being torn about the tear line, wherein each of the pair of wings is selectively removable from the sanitary article about the tear line.

In an embodiment, the outer lateral contour portions comprise compressed material.

In an embodiment, the inner lateral portion of each of the pair of wings comprises compressed material.

In an embodiment, the compressed material is heat sealed.

In an embodiment, the tear line comprises cut lines having relatively longer and short lengths for modulating tensile strength along the tear line.

In an embodiment, the tear line provides for an interdigitating pattern between the outer lateral contour portion of the main body and the inner lateral portion of an adjacent one of the pair of wings along the tear line.

In an embodiment, each of the pair of wings comprises inner tear lines for separating each of the pair of wings into sub-parts thereof.

In accordance with an aspect of the disclosure, there is provided a sanitary article comprising: a longitudinal main body bounded by a perimeter defining opposite ends and opposite lateral margins and comprising a body contacting surface and an opposite outer surface; at least one removable portion extending from at least one segment of the perimeter; and a tear line defined between at least one removable portion and the at least one segment for providing selective detachment therebetween.

In an embodiment of this sanitary article, the at least one removable portion comprises at least one inner tear line thereby providing selective detachment of at least one part thereof therefrom. In an embodiment, the at least one part comprises indicia. In an embodiment, the at least one removable portion comprises a plurality of inner tear lines thereby providing selective detachment of a plurality of parts thereof therefrom. In an embodiment, each of the plurality of parts comprises respective indicia.

In an embodiment of this sanitary article, the at least one segment of the perimeter is positioned at one of the opposite lateral margins of the main body. In an embodiment, the at least one segment of the perimeter is positioned at one of the opposite ends of the main body.

In an embodiment, the sanitary article further comprises a plurality of removable portions, wherein each of the removable portions extends from a respective segment of the perimeter defining a respective tear line therebetween for providing selective detachment therebetween. In an embodiment, one or more of the plurality of removable portions comprises at least one inner tear line thereby providing selective detachment of at least one part thereof therefrom. In an embodiment, the at least one part comprises indicia. In an embodiment, one or more of the plurality of removable portions comprises a plurality of inner tear lines thereby providing selective detachment of a plurality of parts thereof therefrom. In an embodiment, each of the plurality of parts comprises respective indicia. In an embodiment, at least two of the respective segments of the perimeter are respectively positioned at each of the opposite lateral margins of the main body. In an embodiment, at least two of the respective segments of the perimeter are respectively positioned at each of the opposite ends of the main body.

In accordance with another aspect of the present disclosure, there is provided a sanitary article comprising: a longitudinal main body bounded by a perimeter defining opposite ends and opposite lateral margins and comprising a body contacting surface and an opposite outer surface; at least one removable portion extending from at least one segment of the perimeter and comprising at least one inner tear line thereby providing selective detachments of at least one part thereof therefrom.

In an embodiment of the sanitary article in accordance with another aspect of the disclosure, the at least one part comprises indicia. In an embodiment, the at least one removable portion comprises a plurality of inner tear lines thereby providing selective detachment of a plurality of parts thereof therefrom. In an embodiment, each of the plurality of parts comprises respective indicia

In an embodiment of the sanitary article in accordance with another aspect of the disclosure, a tear line is defined between the at least one removable portion and the at least one segment of the perimeter for providing selective detachment therebetween. In an embodiment, the at least one segment of the perimeter is positioned at one of the opposite lateral margins of the main body. In an embodiment, the at least one segment of the perimeter is positioned at one of the opposite ends of the main body.

In an embodiment of the sanitary article in accordance with another aspect of the disclosure, the sanitary article further comprises a plurality of removable portions, wherein each of the removable portions extends from a respective segment of the perimeter, wherein at least two of the plurality of removable portions respectively comprise at least one inner tear line thereby providing selective detachment of at least one respective part thereof therefrom. In an embodiment, the at least one respective part comprises indicia. In an embodiment, one or more of the plurality of removable portions comprises a plurality of inner tear lines thereby providing selective detachment of a plurality of parts thereof therefrom. In an embodiment, each of the plurality of parts comprises respective indicia. In an embodiment, at least two of the respective segments of the perimeter are respectively positioned at each of the opposite lateral margins of the main body. In an embodiment, at least two of the respective segments of the perimeter are respectively positioned at each of the opposite ends of the main body. In an embodiment, a respective lateral tear line is defined between the at least two of the plurality of removable portions and the at least two segments of the perimeter for providing selective detachment therebetween.

In accordance with an embodiment of either one of the above described sanitary articles, the body contacting surface is liquid pervious and/or the outer side of the main body is liquid impervious and/or the outer side is a garment contacting side and/or the outer side comprises an adhesive for adhering to a garment.

In accordance with an embodiment of either one of the above described sanitary articles, the sanitary article further comprises an absorbent core between the body contacting surface and the outer surface.

In accordance with an embodiment of either one of the above described sanitary articles, the removable portion defines inner and outer sides thereof, the removable portion inner side being contiguous with the body contacting surface the removable portion outer side being contiguous with the outer surface.

In accordance with a further aspect of the present disclosure, there is provided a sanitary article comprising: a longitudinal main body defining opposite ends and opposite lateral margins and comprising a liquid pervious body contacting surface, an opposite liquid impervious garment contacting surface and an absorbent core therebetween; and a pair of lateral portions, each lateral portion extending from a respective one of the lateral margins, a lateral tear line defined between at least one of the lateral portions and the respective one of the lateral margins for providing selective detachment therebetween.

In an embodiment of the sanitary article according to this further aspect of the disclosure, another lateral tear line is defined between the other of lateral portions and the respective other one of the lateral margins for providing selective detachment therebetween.

In an embodiment of the sanitary article according to this further aspect of the disclosure, at least one of the lateral portions comprises at least one inner tear line thereby providing selective detachments of at least one part thereof therefrom. In an embodiment, the at least one part comprises indicia. In an embodiment, at least one of the lateral portions comprises a plurality of inner tear lines thereby providing selective detachment of a plurality of parts thereof therefrom. In an embodiment, each of the plurality of parts comprises respective indicia.

In accordance with yet another aspect of the present disclosure, there is provided a sanitary article comprising: a longitudinal main body defining opposite ends and opposite lateral margins and comprising a liquid pervious body contacting surface, an opposite liquid impervious garment contacting surface and an absorbent core therebetween; and a pair of lateral portions, each lateral portion extending from a respective one of the lateral margins, at least one of the lateral portions comprises at least one inner tear line thereby providing selective detachments of at least one part thereof therefrom.

In an embodiment of the sanitary articles in accordance with both of the further and the yet another aspects of the disclosure, the at least one part comprises indicia.

In an embodiment of the sanitary articles in accordance with both of the further and the yet another aspects of the disclosure, at least one of the lateral portions comprises a plurality of inner tear lines thereby providing selective detachment of a plurality of parts thereof therefrom. In an embodiment, each of the plurality of parts comprises respective indicia.

In an embodiment of the sanitary articles in accordance with both of the further and the yet another aspects of the disclosure, at least a lateral tear line is defined between at least one of the lateral portions and the respective one of the lateral margins for providing selective detachment therebetween. In an embodiment, another lateral tear line is defined between the other of lateral portions and the respective other one of the lateral margins for providing selective detachment therebetween.

In an embodiment of the sanitary articles in accordance with both of the further and the yet another aspects of the disclosure, the garment contacting surface comprises an adhesive material for adhering to at least a portion of an undergarment worn by a user. In an embodiment, the lateral portions comprise an adhesive substance for adhering to at least a portion of an undergarment worn by a user. In an embodiment, the lateral portions are selected from the groups consisting of wings, flaps, extensions and combinations thereof.

In accordance with an aspect of the present disclosure, there is provided a sanitary article comprising: an auxiliary body having opposite longitudinal ends, opposite lateral sides, and opposite top and bottom surfaces and being bounded by a perimeter, the auxiliary body defining a central opening through the top and bottom surfaces and comprising a pair of wings, each of the pair of wings extending from a respective lateral side; a main body defining opposite longitudinal ends thereof, opposite lateral sides thereof and comprising a top side, an opposite bottom side and an absorbent core therebetween bounded by a core border formed in the top and bottom sides, the top side defining a body contacting surface within the core border, the bottom side defining a garment contacting surface within the core border, a contour extending beyond the core border and bounded by a sealed perimeter, the contour defining a top contour surface being contiguous with the body contacting surface and an opposite contour undersurface being contiguous with the garment contacting surface, the bottom side of the main body overlying the top surface of the auxiliary body with the garment contacting surface fitted within the opening and the contour undersurface engaging the top surface of the auxiliary body about the opening; and an adhesive substance providing adhesive connection between the contour undersurface and the top surface of the auxiliary body about the opening; wherein the auxiliary body is selectively removable from the main body against the adhesive connection.

In accordance with an aspect of the present disclosure, there is provided a sanitary kit comprising: an auxiliary body having opposite longitudinal ends, opposite lateral sides, and opposite top and bottom surfaces and being bounded by a perimeter, the auxiliary body defining a central opening through the top and bottom surfaces and comprising a pair of wings, each of the pair of wings extending from a respective lateral side; a main body for being mounted to the auxiliary body and defining opposite longitudinal ends thereof, opposite lateral sides thereof and comprising a top side, an opposite bottom side and an absorbent core therebetween bounded by a core border formed in the top and bottom sides, the top side defining a body contacting surface within the core border, the bottom side defining a garment contacting surface within the core border, a contour extending beyond the core border and bounded by a sealed perimeter, the contour defining a top contour surface being contiguous with the body contacting surface and an opposite contour undersurface being contiguous with the garment contacting surface, wherein when mounting the main body to the auxiliary body, the bottom side of the main body overlies the top surface of the auxiliary body with the garment contacting surface being fitted within the opening and the contour undersurface engaging the top surface of the auxiliary body about the opening; and an adhesive substance for providing adhesive connection between the contour undersurface and the top surface of the auxiliary body about the opening; wherein the auxiliary body is selectively removable from the main body against the adhesive connection.

In an embodiment, the contour comprises an inner contour portion near the core border and an outer contour portion near the perimeter, wherein only the contour undersurface of the inner contour portion comprises the adhesive substance providing for no adhesive connection of the contour undersurface of the outer contour portion and the top side of the auxiliary body about the opening. In an embodiment, a downward force on the wings provides a tension force to the main body causing the outer contour portion at the opposite lateral sides of the main body to be lifted.

In an embodiment, the the longitudinal ends of the main body overlie the longitudinal ends of the auxiliary body, wherein the overlying longitudinal ends are free of the adhesive connection providing for the overlying ends to be separated and pulled to peel the main body and the auxiliary body away from each other against the adhesive connection.

In an embodiment, the perimeter of the auxiliary body extends beyond the sealed perimeter of the main body.

In an embodiment, the body contacting surface comprises an adhesive substance for being adhesively connected to a garment.

In an embodiment, each of the pair of wings has a respective top surface and an opposite wing bottom surface comprising an adhesive substance for being adhesively connected to an outer crotch area of an undergarment.

In accordance with an aspect of the present disclosure, there is provided a sanitary article comprising: a main body defining opposite longitudinal ends thereof, opposite lateral sides thereof and comprising a top side, an opposite bottom side and an absorbent core therebetween bounded by a core border formed in the top and bottom sides, the top side defining a body contacting surface within the core border, the bottom side defining a garment contacting surface within the core border, a contour extending beyond the core border and bounded by a sealed perimeter, the contour defining a top contour surface being contiguous with the body contacting surface and an opposite contour undersurface being contiguous with the garment contacting surface, the contour defining opposite lateral contour portions; an auxiliary assembly comprising a pair of separate wing bodies, each of the pair of wing bodies comprises opposite top and bottom surfaces, an inner part and an outer part extending from the inner part, the contour undersurface of each of the contour portions of the main body respectively overlying the top surface of the inner part of each of the wing bodies; an adhesive substance providing adhesive connection between the contour undersurface of the contour portions and the top surface of the inner part of the wing bodies; wherein each of the wing bodies is selectively removable from the main body against the adhesive connection.

In accordance with an aspect of the present disclosure, there is provided a sanitary kit comprising: a main body defining opposite longitudinal ends thereof, opposite lateral sides thereof and comprising a top side, an opposite bottom side and an absorbent core therebetween bounded by a core border formed in the top and bottom sides, the top side defining a body contacting surface within the core border, the bottom side defining a garment contacting surface within the core border, a contour extending beyond the core border and bounded by a sealed perimeter, the contour defining a top contour surface being contiguous with the body contacting surface and an opposite contour undersurface being contiguous with the garment contacting surface, the contour defining opposite lateral contour portions; an auxiliary assembly for being mounted to the main body and comprising a pair of separate wing bodies, each of the pair of wing bodies comprises opposite top and bottom surfaces, an inner part and an outer part extending from the inner part, when mounting the auxiliary assembly to the main body, the contour undersurface of each of the contour portions of the main body respectively overlie the top surface of the inner part of each of the wing bodies; an adhesive substance provides for adhesive connection between the contour undersurface of the contour portions and the top surface of the inner part of the wing bodies; wherein each of the wing bodies is selectively removable from the main body against the adhesive connection.

In an embodiment, the lateral contour portion comprises an inner lateral contour portion near the core border and an outer lateral contour portion near the perimeter, wherein only the contour undersurface of the inner lateral contour portion comprises the adhesive substance providing for no adhesive connection of the contour undersurface of the outer lateral contour portion and the top surface of the inner part of the wing bodies. In an embodiment, a downward force on the wings provides a tension force to the main body causing the outer lateral contour portions of the main body to be lifted.

Other objects, advantages and features of the present disclosure will become more apparent upon reading of the following non-restrictive description of illustrative embodiments thereof, given by way of example only with reference to the accompanying drawings.

### BRIEF DESCRIPTION OF THE DRAWINGS

The following drawings form part of the present specification and present disclosure. In the appended drawings:

FIG. 1 is a top view of the sanitary article in accordance with a non-restrictive illustrative embodiment of the present disclosure;

FIG. 2 is a bottom view of the sanitary article of FIG. 1;

FIG. 3 is a top view of the sanitary article of FIG. 1 showing lateral portions thereof in the process of being removed in accordance with another non-restrictive illustrative embodiment of the present disclosure;

FIG. 4 is a top view of the sanitary article of FIG. 1 showing lateral portions thereof partially removed in accordance with another non-restrictive illustrative embodiment of the present disclosure;

FIG. 5 is a top view of the sanitary article of FIG. 1 showing lateral portions thereof fully removed in accordance with another non-restrictive illustrative embodiment of the present disclosure;

FIG. 6 is a top view of a sanitary article in accordance with a non-restrictive illustrative embodiment of the present disclosure;

FIG. 7 is a top perspective view of a sanitary article in accordance with a non-restrictive illustrative embodiment of the present disclosure and being shown in an assembled position;

FIG. 8 is a top perspective view of the sanitary article of FIG. 7 being shown in a partially disassembled position in accordance with a non-restrictive illustrative embodiment of the present disclosure

FIG. 9 is top perspective view of the main body of the sanitary article of FIG. 7 when disassembled therefrom in accordance with a non-restrictive illustrative embodiment of the present disclosure;

FIG. 10 is a bottom perspective view of the main body of FIG. 9 in accordance with a non-restrictive illustrative embodiment of the present disclosure;

FIG. 11 is top perspective view of the auxiliary body of the sanitary article of FIG. 7 when disassembled therefrom in accordance with a non-restrictive illustrative embodiment of the present disclosure;

FIG. 12 is a bottom perspective view of the auxiliary body of FIG. 11 in accordance with a non-restrictive illustrative embodiment of the present disclosure;

FIG. 13 is a top perspective view of a sanitary article in accordance with a non-restrictive illustrative embodiment of the present disclosure and being shown in a partially disassembled position;

FIG. 14 is a top perspective view of an auxiliary assembly of the sanitary article of FIG. 13 when disassembled therefrom in accordance with a non-restrictive illustrative embodiment of the present disclosure;

FIG. 15 is a front and top perspective view of a sanitary article in accordance with a non-restrictive illustrative embodiment of the present disclosure

FIG. 16 is front and top perspective and sectional view of the sanitary article of FIG. 15 in accordance with a non-restrictive illustrative embodiment of the present disclosure

FIG. 17 is a front perspective and sectional view of the sanitary article of FIG. 15 in accordance with a non-restrictive illustrative embodiment of the present disclosure;

FIG. 18 is a lateral side view of the sanitary article of FIG. 15 in accordance with a non-restrictive illustrative embodiment of the present disclosure;

FIG. 19 is a top and side perspective view of a sanitary article in accordance with a non-restrictive illustrative embodiment of the present disclosure and being shown in an assembled position;

FIG. 20 is another top and side perspective view of a sanitary article in accordance with a non-restrictive illustrative embodiment of the present disclosure and being shown in a assembled position;

FIG. 21 is a side perspective view of a sanitary article in accordance with a non-restrictive illustrative embodiment of the present disclosure and being shown in a assembled position;

FIG. 22 is a top and side perspective view of a sanitary article in accordance with a non-restrictive illustrative embodiment of the present disclosure and being shown in a disassembled position;

FIG. 23 is another top and side perspective view of a sanitary article in accordance with a non-restrictive illustrative embodiment of the present disclosure and being shown in a disassembled position;

FIG. 24 is a bottom and side perspective view of a sanitary article in accordance with a non-restrictive illustrative embodiment of the present disclosure and being shown in a assembled position;

FIG. 25 is a top plan view of a sanitary article in accordance with a non-restrictive illustrative embodiment of the present disclosure;

FIG. 26 is an enlarged view of a portion of FIG. 25 in accordance with a non-restrictive illustrative embodiment of the present disclosure;

FIG. 27 is an enlarged view of a portion of FIG. 25 in accordance with a non-restrictive illustrative embodiment of the present disclosure;

FIG. 28 is an enlarged view of a portion of FIG. 25 in accordance with a non-restrictive illustrative embodiment of the present disclosure;

FIG. 29 is an enlarged view of a portion of FIG. 25 in accordance with a non-restrictive illustrative embodiment of the present disclosure;

FIG. 30 is an enlarged view of a portion of FIG. 25 in accordance with a non-restrictive illustrative embodiment of the present disclosure;

FIG. 31 is an enlarged view of a portion of FIG. 25 in accordance with a non-restrictive illustrative embodiment of the present disclosure;

FIG. 32 is an enlarged view of a portion of FIG. 25 in accordance with a non-restrictive illustrative embodiment of the present disclosure;

FIG. 33 is a top plan view of a sanitary article including lateral wings in accordance with a non-restrictive illustrative embodiment of the present disclosure;

FIG. 34 is a top plan view of the sanitary article of FIG. 33 with the lateral wings having been removed in accordance with a non-restrictive illustrative embodiment of the present disclosure;

FIG. 35 is a bottom view of the sanitary article of FIG. 34 including a film covering adhesive material in accordance with a non-restrictive illustrative embodiment of the present disclosure; and

FIG. 36 is another bottom view of the sanitary article of FIG. 35 with the film having been removed exposing the adhesive material in accordance with a non-restrictive illustrative embodiment of the present disclosure.

### DETAILED DESCRIPTION

Generally stated and in accordance with an aspect of the present disclosure, there is provided a there is provided a sanitary article comprising a longitudinal main body bounded by a perimeter defining opposite ends and opposite lateral margins and comprising a body contacting surface and an opposite outer surface. At least one removable portion extends from at least one segment of the perimeter. In an embodiment, a tear line is defined between at least one removable portion and the at least one segment for providing selective detachment therebetween. In an embodiment, the at least one removable portion comprises at least one inner tear line thereby providing selective detachments of at least one part thereof therefrom

Generally stated and in accordance with an aspect of the present disclosure, there is provided a sanitary article comprising a longitudinal main body defining opposite ends and opposite lateral margins and comprising a liquid pervious body contacting surface, an opposite liquid impervious garment contacting surface and an absorbent core therebetween. A pair of lateral portions respectively extend from a respective one of the lateral margins. In an embodiment, a lateral tear line is defined between at least one of the lateral portions and the respective one of the lateral margins for providing selective detachment therebetween. In an embodiment, at least one of the lateral portions comprises at least one inner tear line thereby providing selective detachments of at least one part thereof therefrom.

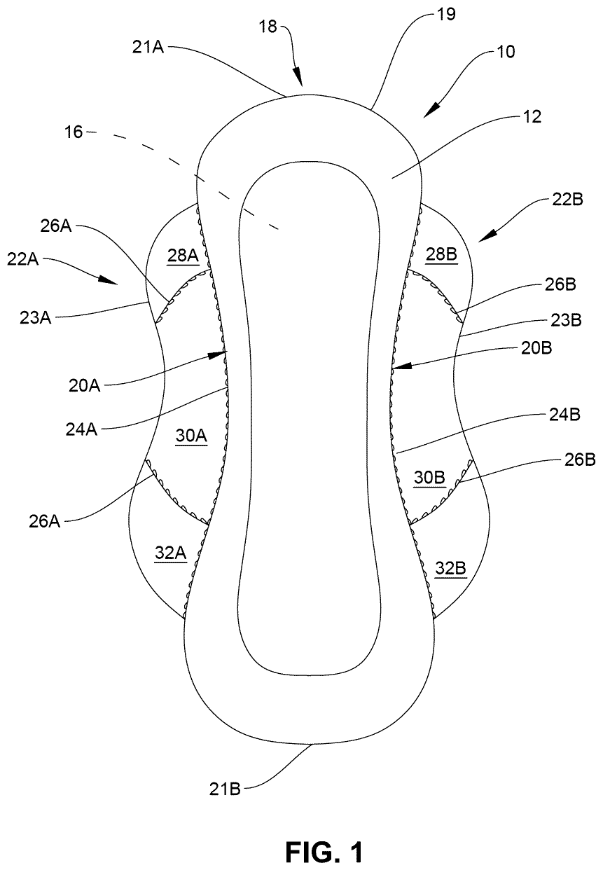

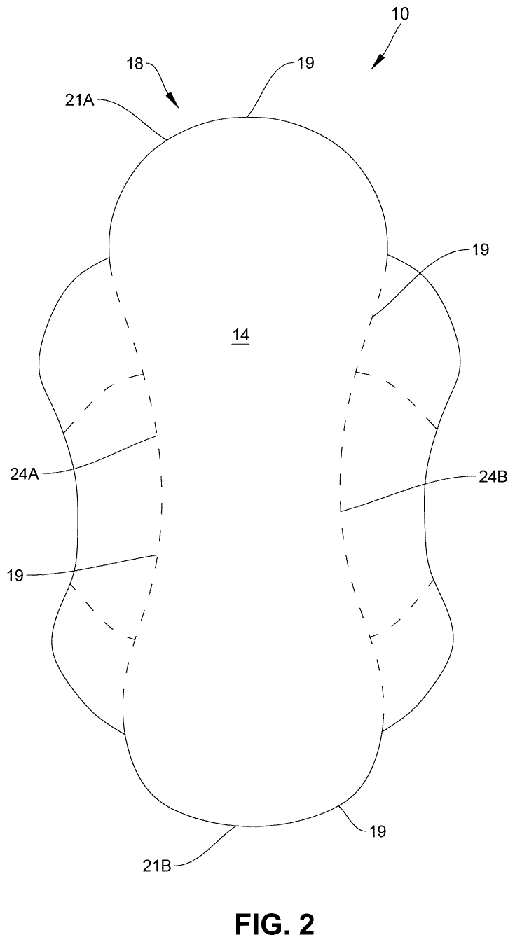

FIGS. 1 and 2 show the sanitary article 10, including a top surface 12 sheet 12, a backing sheet 14 and core 16 therebetween.

In an embodiment, the top sheet 12 is liquid pervious and defines the body contacting side or surface of the sanitary article 10. The backing sheet 14 is liquid impervious thereby acting as a protective barrier. The backing sheet 14 defines an outer side such as a garment (or undergarment) contacting side or surface. In an embodiment, this surface adheres to the wearer's undergarment via an adhesive. Adhesives such as pressure sensitive adhesives are suitable for this purpose. The core 16 is an absorbent core that absorbs discharge. In an embodiment the body contacting surface of the sanitary article 10 may also include an adhesive to adhere to the body of the user.

The top sheet 12 is made of materials which are compliant and readily conform to the shape of the body. The top sheet 12 may exhibit good strikethrough and rewet characteristics, permitting bodily discharges to rapidly penetrate therethrough to the core 16, but not flow back to the skin of the wearer. The top sheet 12 may be porous to permit discharged liquids to pass through to the core 16. The body contacting surface provides comfortable and dry-feeling contact with body surfaces while allowing free passage of fluids therethrough into the absorbent core 16.

The backing sheet 14 prevents discharges absorbed by the core 16, from escaping. The backing sheet 14 prevents the fluids which are expelled, or which escape from the absorbent core 16 from soiling the user's garments.

The absorbent core 16 is intermediate the top sheet 12 and the backing sheet 14 and the article 10 is integrally or unitarily assembled. Therefore, the body contacting surface of the article 10 is defined by the top sheet 12 and the opposite protective barrier or garment contacting surface is defined by the backing sheet 14. The absorbent core 16 has two opposed faces, one oriented towards the backing sheet 14 and one oriented towards the top sheet 12. The absorbent core 16 provides for receiving, collecting and containing bodily discharges such as menses, deposited thereon or which pass through the top sheet 12. In an embodiment, the core 16 is configured to be narrow and thin so as to be comfortable to the wearer.

The article 10 defines a longitudinal main body 18 comprising the top sheet 12, the backing sheet 14 and the absorbent core 16. The main body 18 is delimited or bounded by a perimeter 19 that defines opposite lateral sides or margins 20A and 20B and opposite ends 21A and 21B. The opposite lateral margins 20A and 20B run along the longitudinal length of the main body 18. The opposite lateral margins 20A and 20B define the width of the body portion 18 whereas the ends 21A and 21B define the length of the body portion 18.

The article 10 comprises such as lateral portions such as a pair of wings or flaps 22A and 22B which extend beyond each margin 20A and 20B. Each later portion 22A and 22B respectively and define a respective outer free edge 23A and 23B.

The lateral portions 22A and 22B may extend along the longitudinal length or segment of each margin 20A and 20B. The lateral portions 22A and 22B may be contiguous with the backing sheet 14, contiguous with the top sheet 12 or a laminate of both the top sheet 12 and the backing sheet 14. Each lateral portion 22A and 22B has a garment contacting face or portion that contacts the wearer's undergarments in use which may also include an adhesive material. The lateral portions 22A and 22B may be provided in a variety of suitable shapes and when in use extend beyond the undergarment crotch portion to be flipped thereunder and adhered to the outer surface of the undergarment opposite the body contacting surface thereof.

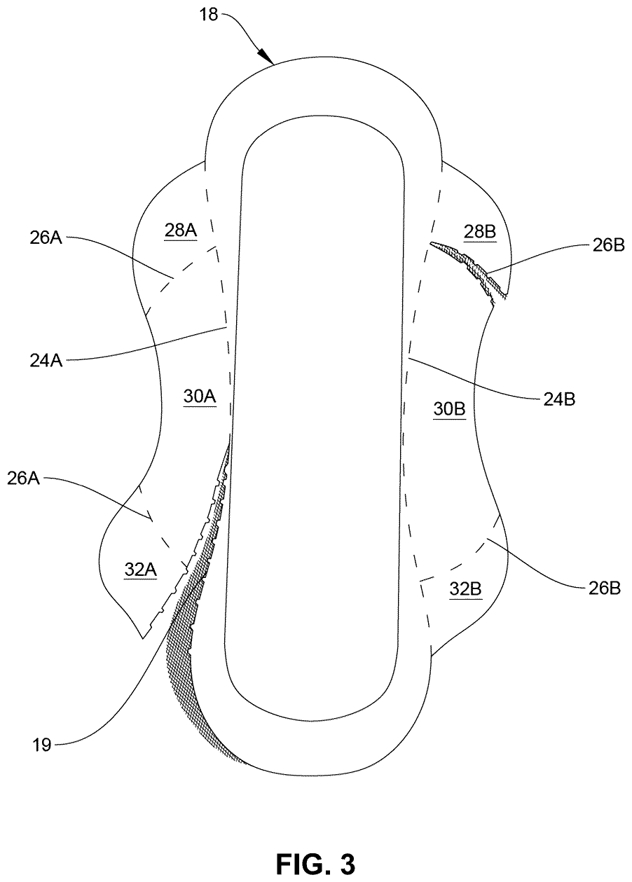

With reference to FIGS. 1, 2 and 3, a respective tear line 24A and 24B is formed between each lateral portions 22A and 22B and its respective adjacent margin 20A and 20B providing the user to fully remove each lateral portion from the main body portion 18 as shown in FIG. 5. The tear line 24A or 24B provides the user with detaching the lateral portion 22A or 22B from along the length of the tear line 24A or 24B without disturbing the physical integrity of the margins 20A or 2B. Thus, when the lateral portions 22A and 22b are fully removed, the margins 20A and 20B are left free defining the lateral edges of the article

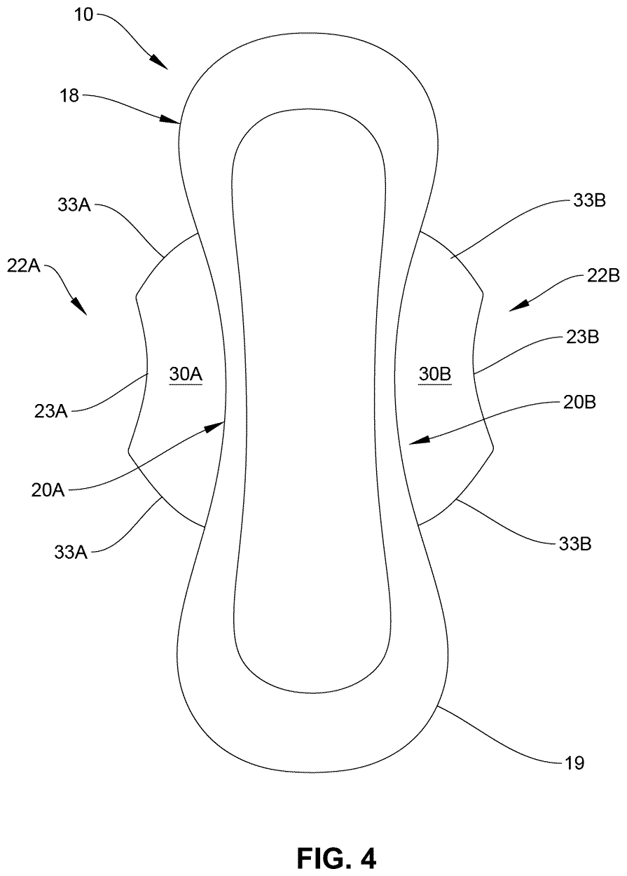

Respective tear lines 26A and 26B are formed within each lateral portion 22A and 22B thereby defining parts or sub-parts thereof. In this example, lateral portion 22A defines three separately removable parts, 28A, 30A and 32A formed between tear lines 24A, 26A and the outer edge 23A. Similarly, lateral portion 22B defines three separately removable parts, 28B, 30B and 32B formed between tear lines 24B, 26B and the outer edge 23B. Each part 28A, 30A and 32A can be selectively and separately removed from lateral portion 22A. Similarly, each part 28B, 30B and 32B can be selectively and separately removed from lateral portion 22B. For example, FIG. 4 shows that parts 28A and 32A have been removed from lateral portion 22A and parts 28B and 32B have been removed from lateral portion 22B, leaving parts 30A and 30B extending from the margins 20A and 20B and defining a remaining portions of the outer edges 23A and 23B as well as free edges 33A and 33B, respectively.

A variety of suitable sizes and configurations for each lateral portion 22A and 22B can be provided within the context of the present description. Moreover, the lateral portions 22A and 2B can be further divided via additional tear lines therein into a varying number of parts with variety of suitable sizes and configurations.

As such, a user may selectively remove either one or both of the lateral portions 22A and 22B. A user may selectively remove parts of one or both of the lateral portions 22A and 22B.

The parts of the lateral portions 22A and 22B may also include indicia in order to prompt the user to remove the parts so as to modulate the size or the configuration of the lateral portions as desired or as required.

Considering various activities of a user, she may choose a sanitary pad with or without wings or with bigger or smaller wings or differently shaped wings thus the tear lines. The present sanitary article provides these options without recourse to different articles to meet different needs dependent on various activities. Moreover, with respect to incontinence issues some women prefer no wings, this is also true for some in the summer when dressed in lighter clothing. As such, the present sanitary articles provide these options based on user preferences.

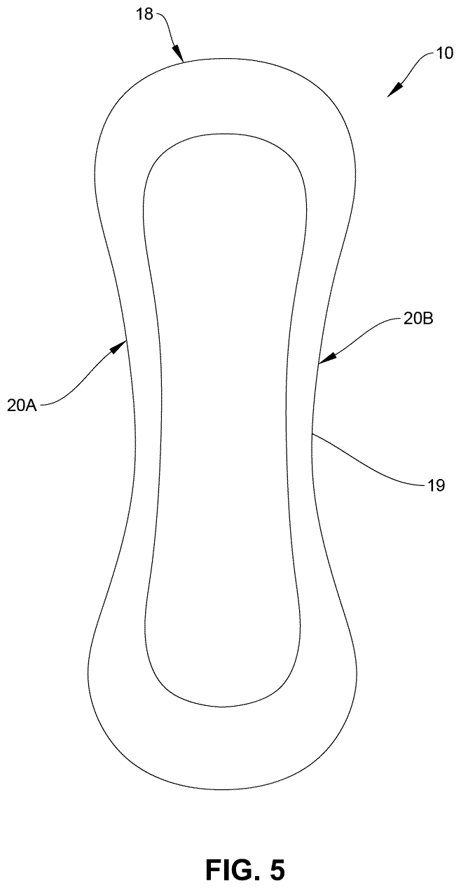

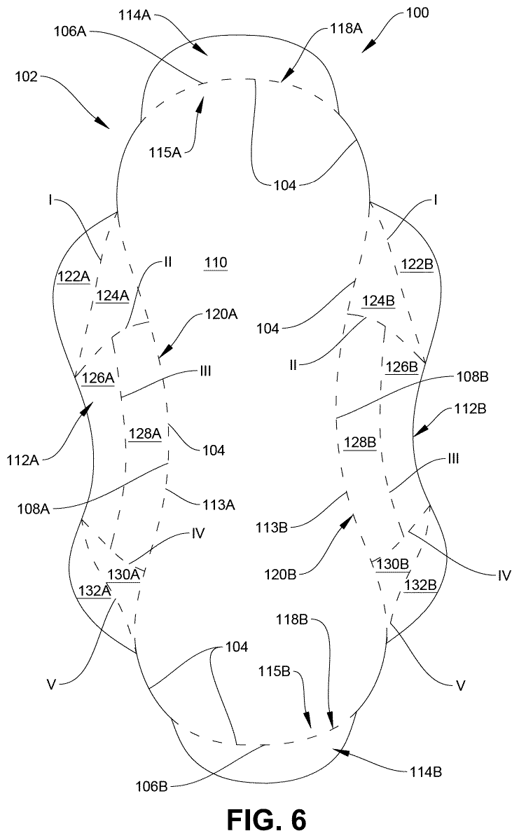

Turning now to FIG. 6, there is shown a sanitary article 100, which can be an incontinence article or a diaper for example. Article 100 is similar but not identical to article 10 and comprises a main longitudinal body 102 delimited or bounded by a perimeter 104. The perimeter 104 defines opposite longitudinal ends or end margins 106A and 106B and opposite lateral sides or lateral margins 108A and 108B. The main body 102 defines a body contacting surface 110 and an opposite outer surface (not shown but similar to surface 14 for article 10).

Removable portions 112A, 112B, 114A, 114B respectively extend from segments 113A, 113B, 115A, 115B of the perimeter 104. Segments 113A and 113B are positioned at respective lateral sides or lateral margins 108A and 108B. Segments 115A and 115B are positioned at respective longitudinal ends or end margins 106A and 106B. Tear line 118A is defined between the removable portion 112A and the segment 113A for selective detachment therebetween. Tear line 118B is defined between the removable portion 112B and the segment 113B for selective detachment therebetween. Tear line 120A is defined between the removable portion 114A and the segment 115A for selective detachment therebetween. Tear line 120A is defined between the removable portion 113A and the segment 115A for selective detachment therebetween. In the present example, removable portions 112A and 112B are lateral portions and removable portions 114A and 114B are end portions. In an embodiment, the article 100 includes only one removable portion. In an embodiment, the article 100 includes more removable portions extending from segments of the perimeter 104 than those illustrated in FIG. 6.

In the example shown here, removable portions 112A and 112B include respective inner tear lines I, II, III, IV and V defining respective parts thereof. In this example, removable portion 112A includes removable parts 122A, 124A, 126A, 128A, 130A, and 132A and removable portion 112B includes removable parts 122B, 124B, 126B, 128B, 130A, and 132B. In an embodiment, article 100 does not include lateral tear lines 118A and 118B and only includes the inner lines in order to provide removing selective parts of the removable portions.

In an embodiment, the removable portions and removable parts of sanitary article 100 include indicia for providing the user to selectively remove portions and/or parts based on suggested configurations or preference.

Sanitary article 100 can be provided in a variety of suitable sizes and configurations.

The skilled artisan will readily appreciate that tear lines include without limitation weak lines, perforated lines which may or may not include dotted lines for allowing the user to tear the portions at each side of the tear line along the dotted line as is known in the art.

Generally stated and in accordance with an aspect of the present disclosure, there is provided a sanitary article comprising an auxiliary body having opposite longitudinal ends, opposite lateral sides, and opposite top and bottom surfaces and being bounded by a perimeter. The auxiliary body defines a central opening through the top and bottom surfaces and comprises a pair of wings. Each of the wings extends from a respective lateral side. A main body defines opposite longitudinal ends thereof, opposite lateral sides thereof and comprises a top side, an opposite bottom side and an absorbent core therebetween bounded by a core border formed in the top and bottom sides. The top side defines a body contacting surface within the core border. The bottom side defines a garment contacting surface within the core border. A contour extends beyond the core border and is bounded by a sealed perimeter. The contour defines a top contour surface being contiguous with the body contacting surface and an opposite contour undersurface being contiguous with the garment contacting surface. The bottom side of the main body overlies the top surface of the auxiliary body with the garment contacting surface fitted within the opening and the contour undersurface engaging the top surface of the auxiliary body about the opening. An adhesive substance provides adhesive connection between the contour undersurface and the top surface of the auxiliary body about the opening. The auxiliary body is selectively removable from the main body against the adhesive connection.

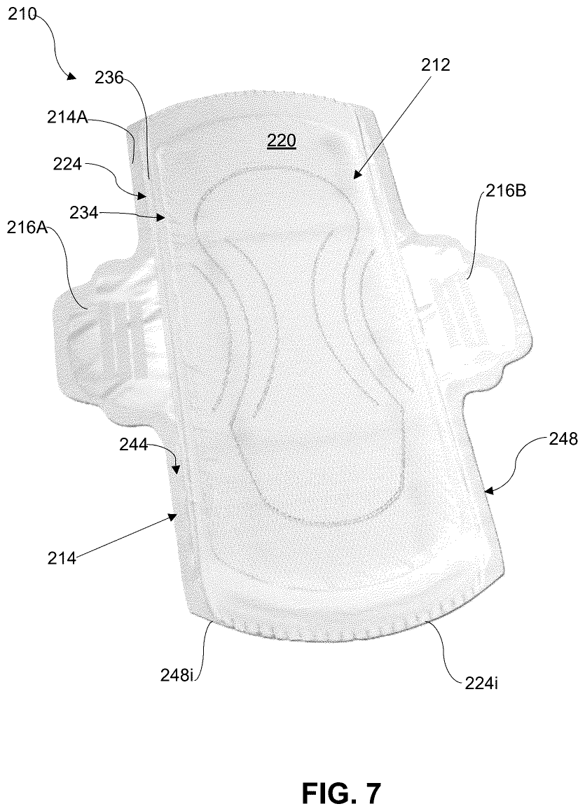

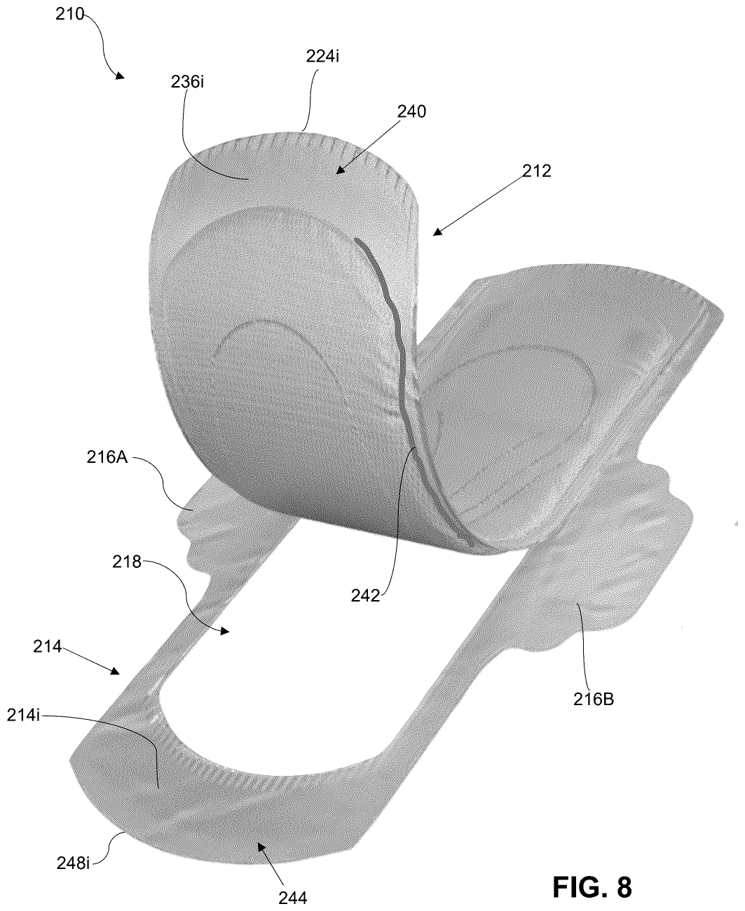

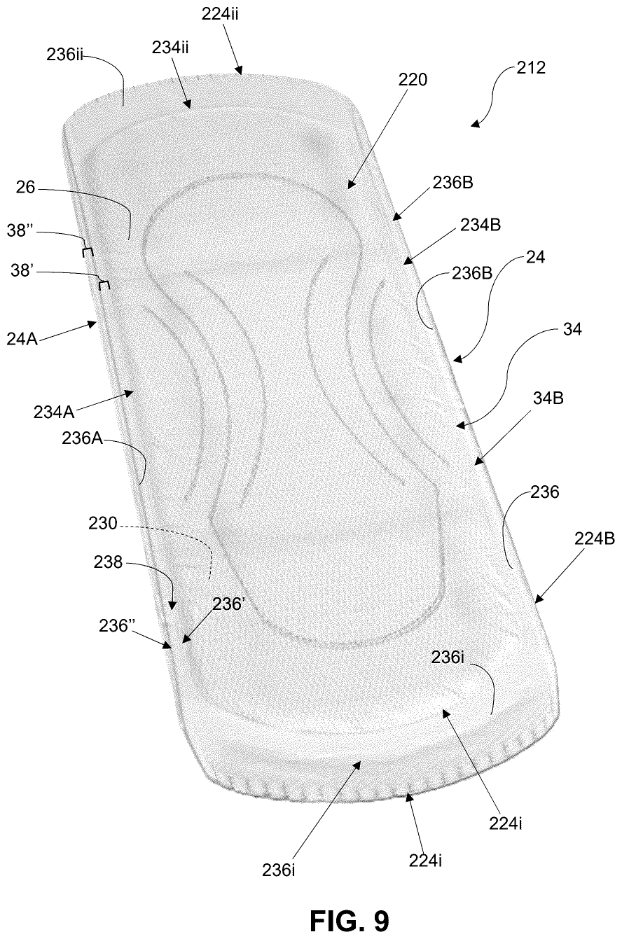

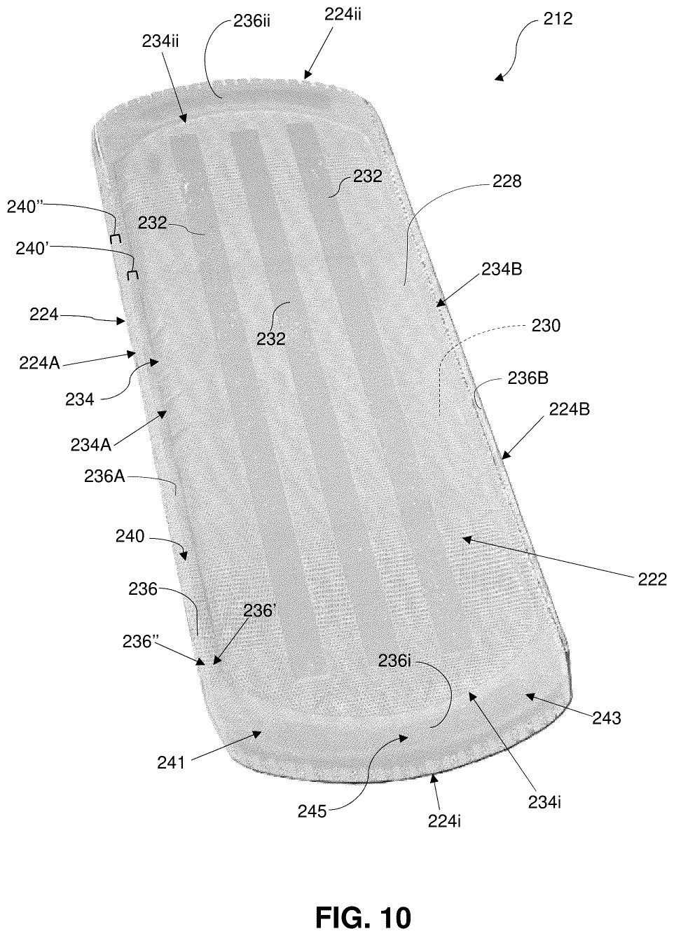

With reference to FIGS. 7-12, a sanitary article 210 in accordance with a non-restrictive illustrative embodiment of the present disclosure includes a main body 212 and an auxiliary body 214 adhesively assembled together. FIG. 7 shows the sanitary article 210 in an assembled configuration including lateral extending portions or wings 216A and 216B. As shown in FIG. 8, the main body 212 is peeled off the auxiliary body 214 (against the adhesive force) disassembling bodies 212 and 214 thereby providing a wingless sanitary article 212 shown in FIGS. 9 and 10. The auxiliary body 214 which can now be discarded maintains the wings 216A and 216B and defines an opening 18 for receiving the main body 212.

Thus, the main body 212 is either as standalone wingless sanitary article 212 as shown in FIGS. 9 and 10, or a component of a winged sanitary article assembly 210 as shown in FIG. 7.

Turning to FIGS. 9 and 10, the main body 212 defines a top side 220, a bottom side 222 and a perimeter 224. The main body 212 is made of laminated sheet including a top sheet 226 at the top side, an opposite bottom or backing sheet 228 at the bottom side and an absorbent core 230 sandwiched therebetween.

The top sheet 226 defines the top surface of sanitary articles 210 and 212 and is a body contacting surface that is liquid previous to allow flow of discharge towards the absorbent core 230 whereas the backing sheet 228 defines a garment (or undergarment) contacting surface that is liquid impervious thereby acting as a protective barrier. The backing sheet 228 includes adhesive strips 232 for adhering to the wearer's undergarment.

The main body 212 defines an inner seal line 234 that is the border line of the absorbent core 230 and is recessed from the sealed perimeter line 224 defining a contour portion 236 therebetween. The border line 234 defines lateral borders or margins 234A and 234B and front and rear margins 234i and 234ii. The sealed perimeter line 224 defines lateral sealed lines 224A and 224B and front and rear sealed lines 224i and 224ii. The contour portion 236 comprises sheet material extending between the border line 234 and the perimeter line 224. The contour portion 236 defines opposite lateral portions 236A and 236B and front and rear portions 236i and 236ii Lateral contour part 236A extends between lateral margin 234A and lateral sealed line 224A. Lateral contour part 236B extends between lateral margin 234B and lateral sealed line 224B. Front contour part 236i extends between front margin 234i and front sealed line 224i. Rear contour part 236ii extends between rear margin 234ii and rear sealed line 224ii. The contour portion 236 defines a top surface 238 at top side 220 and a bottom or undersurface 240 at bottom surface 222. The contour portion 236 defines an inner part 236′ near the border line 234 and an outer part 236″ near the perimeter line 224. The inner part 236′ defines a top surface inner portion 238′ and an undersurface inner portion 240′. The outer part 234″ defines a top surface outer portion 238″ and an undersurface outer portion 240″.

The undersurface 240 comprises an adhesive substance 242 (see FIG. 8). In an embodiment, the adhesive substance 242 is provided only on the inner undersurface portion 240′ not on the outer undersurface portion 240″. In an embodiment, the adhesive substance 242 is provided only on the undersurface 240 of the lateral contour parts 236A and 236B and not on the undersurface 240 of the front and rear contour parts 236i and 236ii. In an embodiment, the adhesive substance 242 is provided on lateral sides 241, 243 of the undersurface 240 of the front and rear contour parts 236i and 236ii. In an embodiment, the lateral sides 241, 243 respectively flank a central part 245 therebetween without any adhesive substance. In an embodiment, the foregoing is only provided at the front lateral contour part 236i. In an embodiment, the undersurface 240 of the front contour part 36i does not include an adhesive substance.

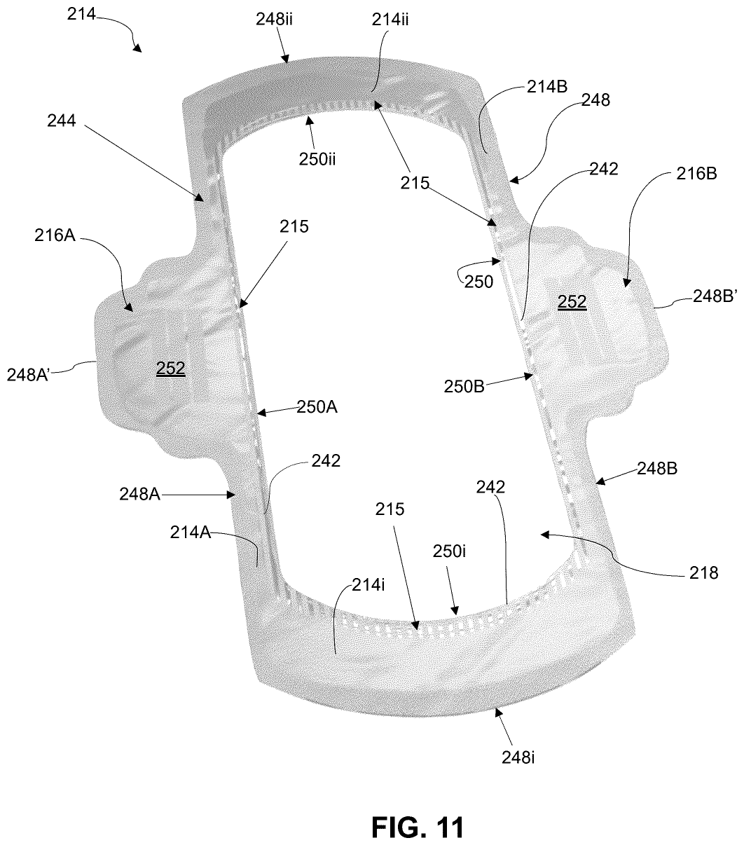

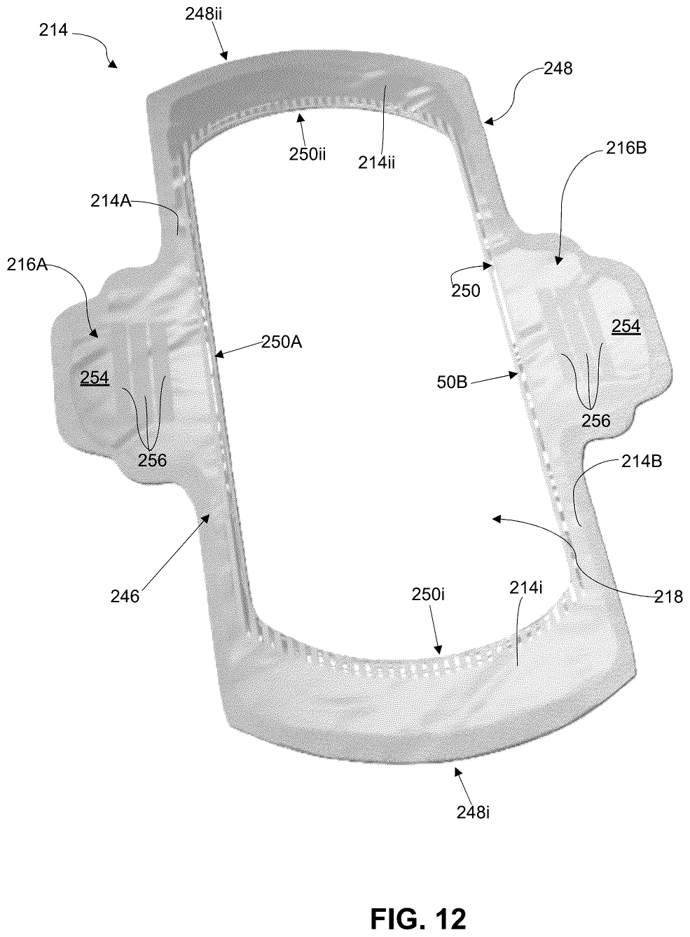

Turning to FIGS. 11 and 12, the auxiliary body 214 defines opposite top and bottom side 244 and 246, respectively and a sealed perimeter line 248 and further defines a central opening 218 for the main body 212. The perimeter line 248 is the perimeter of the fully assembled sanitary article 210 as shown in FIG. 7 and defines lateral perimeter edges 248A and 248B and front and rear perimeter edges 248′ and 248″. The opening 218 is circumscribed by an inner rim 250 defining lateral rim edges 250A and 250B, respectively, and front and rear rim edges 250i and 250ii, respectively. The auxiliary body 214 defines lateral body portions 214A and 214B and front and rear body portions 214i and 214ii. The wings 216A and 216B respectively extend from the lateral body portions 214A and 214B and the perimeter lateral perimeter edges 248A, 248B respectively include portions thereof 248A′ and 248B′ that defines the free edges of the wings 216A and 216B. The wings 216A and 216B define respective top surfaces 252 and respective bottom surfaces 254 which include adhesive strips 256.

The auxiliary body 214 includes an adhesive substance 242 on its top surface 244 about the opening 218. In an embodiment, the top surface at the front body portion 214i has lateral parts 255 and 257 that flank a central part 259 therebetween. In an embodiment, the lateral parts 255 and 257 include the adhesive substance 242 and the central part 259 does not include the adhesive substance. In an embodiment, the top surface 244 at the front body portion 214i does not include any adhesive substance. In an embodiment, the front body portion 214i only includes an adhesive substance 243 along a line 215 near the rim 250. Indeed, the adhesive line 215 can fully circumscribe the rim 250 and as such the rest of the top surface 244 of the auxiliary body is not in adhesive engagement with

With reference to FIGS. 7-12, when the main body 212 is assembled with the auxiliary body 214 the undersurface 240 of the contour part 236 overlies the top side 244 of auxiliary body 214 about the opening 218. More specifically, the undersurfaces 240 of contour parts 236A, 236B, 236i, 236ii respectively overly the top sides of the auxiliary body parts 214A, 214B, 214i, 214ii. The inner margin 224 at the undersurface 240 of the main body 214 is substantially aligned with the rim 250 providing for the opening 218 to expose the bottom side 222 of the main body 214.

In an embodiment, the absence of an adhesive substance 242 (previously described above) in a front area of the sanitary article 210 between the undersurface 240 of the front contour part 236i and the top side 244 of the front auxiliary body part 214i, the front edges 224i and 248i (see FIGS. 7 and 8) can be opened by pulling apart parts 236i and 214i thereby progressively peeling apart the main body 212 and the auxiliary body 214 against the adhesive force and as such providing for a wingless sanitary article 212. It should be noted that the foregoing is applicable to the rear part of the sanitary article 210 and thus the same absence between undersurface 240 of the rear contour part 236ii and the top side 244 of the rear auxiliary body part 214ii provides for creating rear flaps which allows the user to open edges 224ii and 248ii. In an embodiment, the terms “rear” and “front” are interchangeable as the sanitary article 210 can be positioned for use in either direction which then (when in use) defines the front and rear of the article 10.

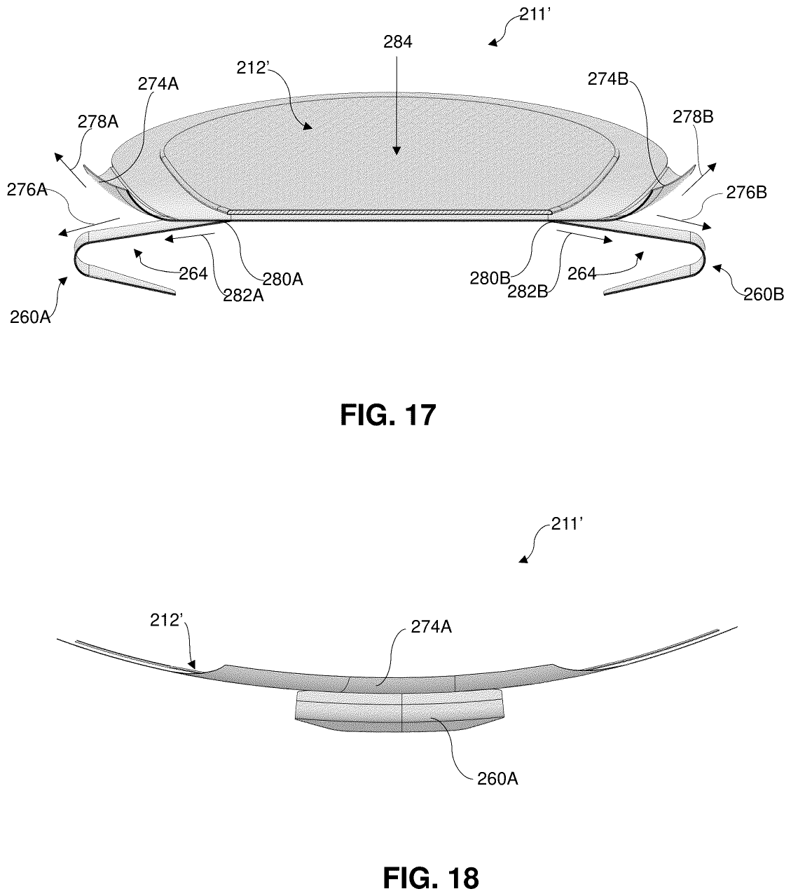

In an embodiment and as exemplified in FIG. 8, the inner undersurface part 240′ of the lateral contour parts 236A and 236B include the adhesive substance 242. Similarly, the adhesive substance 242 is provides on an inner portion of the top side 244 of the lateral body parts 214A and 214B near the rim edges 250A and 250B such that the outer undersurface parts 240″ of the of the lateral contour parts 236A and 236B rest on the top sides 244 of the lateral body parts 214A and 214B without any adhesive therebetween. The foregoing provides an additional sheet of material of the main body 212 extending from the adhesively connected junction between the main body 212 and auxiliary body 214 and overlying the auxiliary body 214. This additional sheet of material will be further discussed with reference to FIGS. 12-18 hereinbelow.

Generally stated and in accordance with an embodiment, there is provided a sanitary article comprising a main body defining opposite longitudinal ends thereof, opposite lateral sides thereof and comprising a top side, an opposite bottom side and an absorbent core therebetween bounded by a core border formed in the top and bottom sides. The top side defines a body contacting surface within the core border. The bottom side defines a garment contacting surface within the core border. A contour extends beyond the core border and is bounded by a sealed perimeter. The contour defines a top contour surface being contiguous with the body contacting surface and an opposite contour undersurface being contiguous with the garment contacting surface. The contour defines opposite lateral contour portions. An auxiliary assembly comprises a pair of separate wing bodies. Each of the pair of wing bodies comprises opposite top and bottom surfaces, an inner part and an outer part extending from the inner part. The contour undersurface of each of the contour portions of the main body respectively overlies the top surface of the inner part of each of the wing bodies. An adhesive substance provides adhesive connection between the contour undersurface of the contour portions and the top surface of the inner part of the wing bodies. Each the wing body is selectively removable from the main body against the adhesive connection.

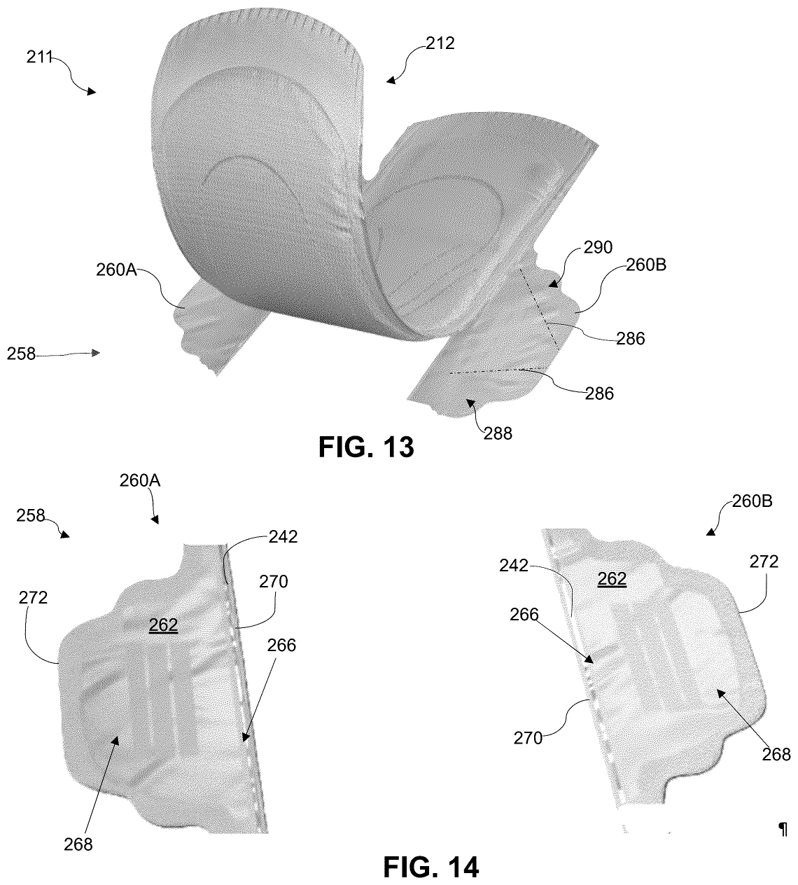

With reference to FIGS. 13-14, there is shown a sanitary article 211, in accordance with another illustrative embodiment, comprising the main body 212 (previously described) and an auxiliary assembly 258 comprising a pair of separate wing bodies 260A and 260B. Each of the wing bodies 260A and 260B respectively comprises a top surface 262 and an opposite bottom surface 264 (see FIG. 18) which includes an adhesive substance for adhesively engaging the outer part of the crotch area of an undergarment. Each of the wing bodies 260A and 260B respectively comprises an inner part 266 and an outer part 268 extending from the inner part 266. The inner part 266 defines an inner free edge 270 and the outer part 266 defines an outer free edge 272. The inner part 266 comprises an adhesive substance 242 at the top surface 262 thereof for adhesive engagement with the inner undersurface part 40′ at the lateral contour parts 236A and 236B. As such, the wings 260A and 260B can be removed (see FIG. 14) providing the wingless sanitary article 212 as shown in FIGS. 9 and 10.

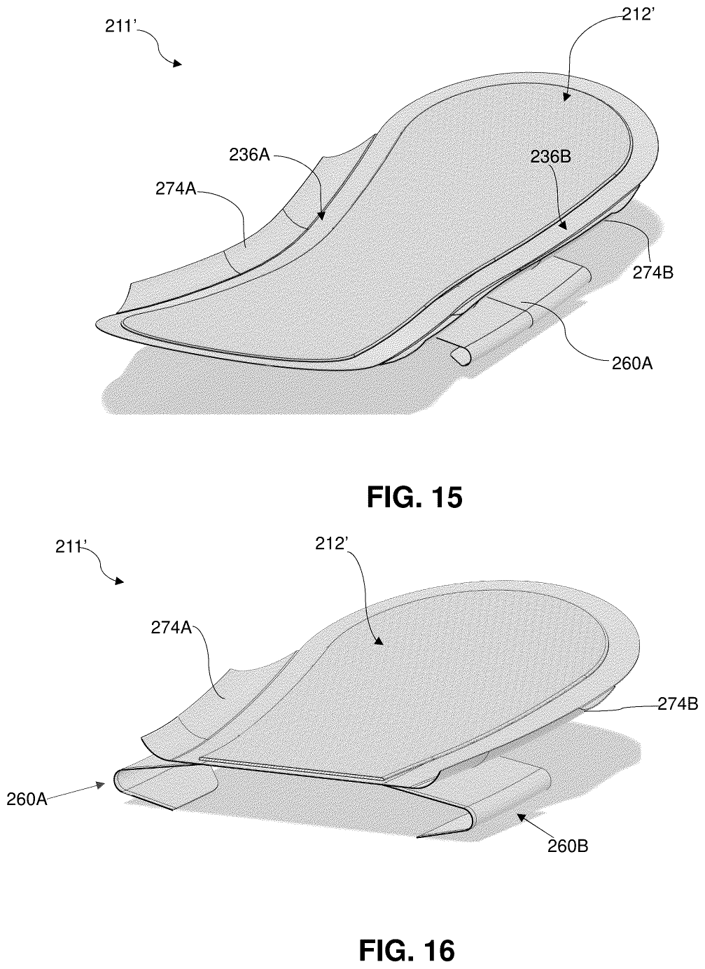

With reference to FIGS. 15-18, there is shown a sanitary article 211′, in accordance with another illustrative embodiment, comprising a main body 212′ (similar to body 212) and wing bodies 260A and 260B. The outer undersurface parts 240″ of the lateral contour parts 236A and 236B of the main body 212′ are not in adhesive contact with the top sides 262 of the wing bodies 260A and 260B thereby providing lateral portions 274A and 274B that freely overly the wing bodies 260A and 260B respectively. These lateral portions 274A and 274B are similar (but shorter in length) to the above referenced additional sheet of material of the main body 212 extending from the adhesively connected junction between the main body 212 and auxiliary body 214 and overlying the auxiliary body 214. When the user pulls the wings 260A and 260B downwardly as respectively shown by arrows 276A and 276B to curve them inwardly so that their bottom surfaces 264 adhere to the undergarment, these free lateral portions 274A and 274B are forced upwardly as respectively shown by arrows 278A and 278B due to the tension in the main body 212 caused at each lateral junction 280A and 280B. The junction 280A is the area where there is adhesive connection between the inner undersurface part 240′ of the lateral contour part 236A and the top side 262 of the inner part 266 of the wing body 260A. The junction 280B is the area where there is an adhesive connection between the inner undersurface part 240′ of the lateral contour part 236B and the top side 262 of the inner part 266 of the wing body 260B. When the wing bodies 260A and 260B are pulled downwardly as shown by arrows 276A and 276B, the junctions 280A and 280B are pulled outwardly and downwardly as shown by arrows 282A and 282B and the tension caused to the main body 212 provides for the lateral portions 274A and 274B to be lifted or forced upwardly as shown by arrows 278A and 278B providing lateral barriers and forming a cup like configuration 284 at the user engaging side of the article 211′.

Turning back to FIG. 13, the wing bodies 260A and 260B may include tear lines 286 for removing portions 288 and 290 to decrease the size of a wing body 260A and 260B.

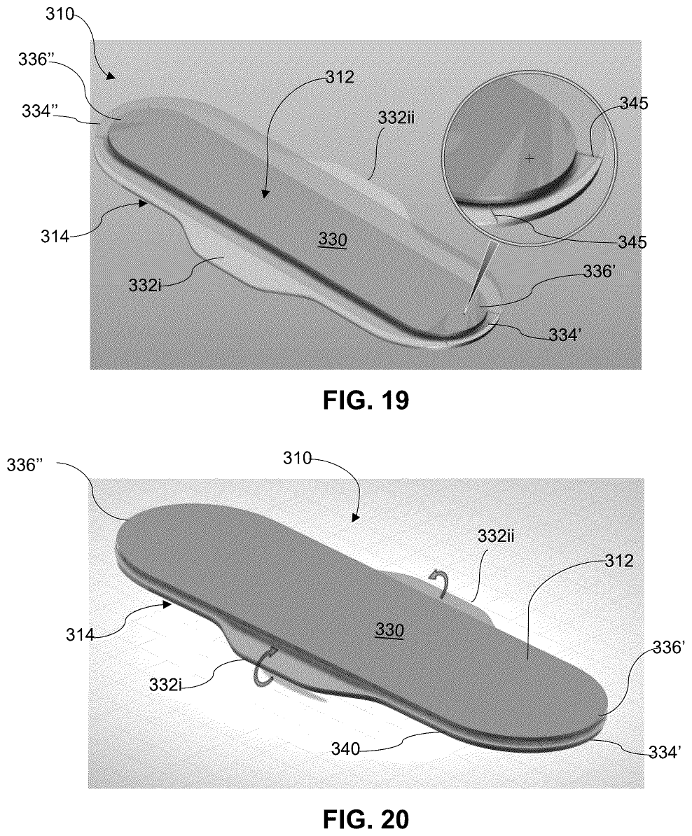

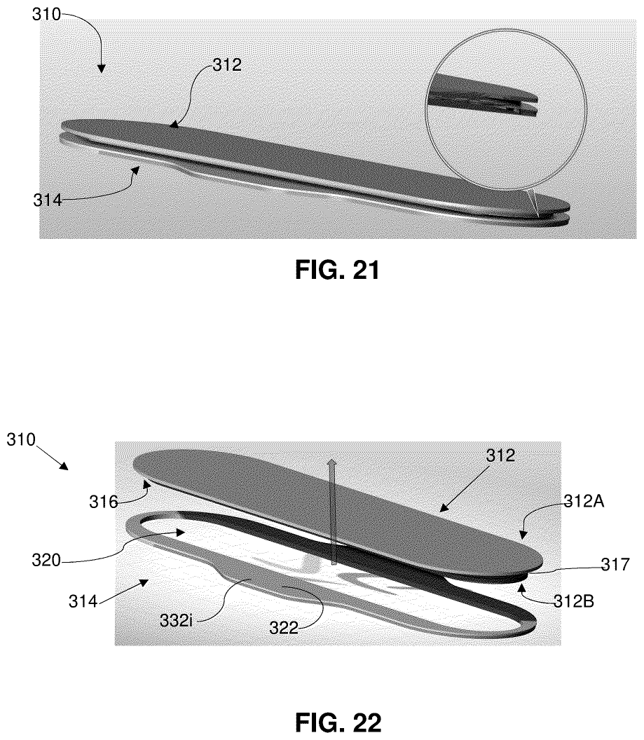

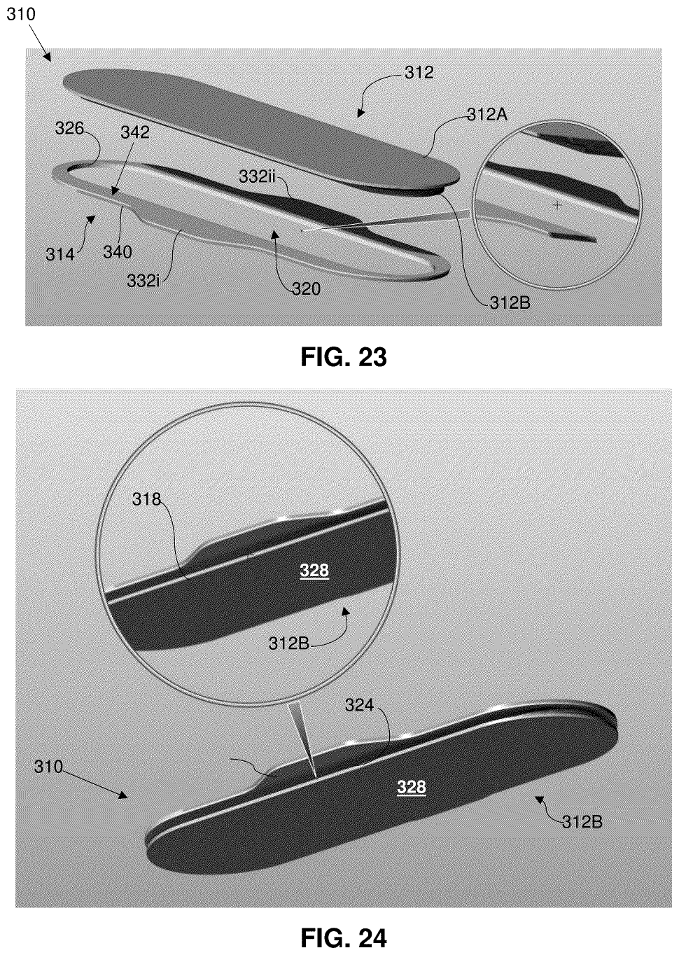

With reference to FIGS. 19-24, a sanitary article 310 in accordance with a non-restrictive illustrative embodiment of the present disclosure is described, including a main body 312 and an auxiliary body 314.

The main body 312 comprises an upper body portion 312A and a lower body portion 312B. The lower body portion 312B is recessed compared to the upper body portion 312A. As such, the upper body portion 312A defines an undersurface 316 circumscribing the lower body portion 312B. The lower body portion 312B defines a peripheral wall 318 downwardly extending from the undersurface 316 forming a shoulder 317 therebetween.

The auxiliary body 314 comprises a ring structure defining a central opening 320, a top surface 322 and a bottom surface 324. In an embodiment, the undersurface 316 overlies the top surface 322 and is removably secured thereto via an adhesive therebetween. The opening 320 receives the lower body portion 312B therethrough. The opening 320 is defined by a rim wall 326 formed at a junction between the top and bottom surfaces 322 and 324, respectively. In an embodiment, the rim wall 326 engages the peripheral wall 318 of the lower body portion 312B. In an embodiment, the rim wall 326 is removably secured to peripheral wall 318 via an adhesive therebetween. In an embodiment, the undersurface 316 is spaced from the top surface 322 and the adhesive connection between the top and bottom body portion 312A and 312B is provided by the contact between walls 326 and 318. The lower body portion 312B defines an undersurface or bottom surface 328 and the upper body portion 312A defines an opposite top surface 330 thereof. The lower body undersurface 328 is flush with or extends downwardly of the bottom surface 324 of the auxiliary body 314.

The auxiliary body 314 comprises wing elements 332i and 332ii laterally extending therefrom.

In an embodiment, then the sanitary article is assembled, the front and rear ends of the auxiliary body 314, respectively denoted by reference numerals 334′ and 334″, respectively extend beyond the front and rear ends of the main body 312 respectively denoted by reference numerals 336′ and 336″.

The user may selectively peel off the auxiliary body 314 from the main body 312 against the adhesive force therebetween thereby disassembling the sanitary article 310. In this way the user removes portions from the sanitary article 310 that are integral to the auxiliary body 314, such as the lateral wing elements 332i and 332ii or the front and rear ends 136′ and 136″. When the auxiliary body 314 is removed, the remaining main body 312 defines the sanitary article 310, thus the sanitary article 310 is the main body 312.

In an embodiment, the top surface 330 of the upper body portion 312A comprises a top sheet that is liquid pervious and defines the body contacting side or surface of the sanitary article 310. In an embodiment, the bottom surface 28 of the lower body 312B comprises a backing sheet that is liquid impervious thereby acting as a protective barrier. The backing sheet 328 defines an outer side such as a garment (or undergarment) contacting side or surface. In an embodiment, this surface adheres to the wearer's undergarment via an adhesive. Adhesives such as pressure sensitive adhesives are suitable for this purpose. A core is positioned within the junction of the upper and lower bodies, 312A and 312B respectively. The core is an absorbent core that absorbs discharge. In an embodiment the body contacting surface of the sanitary article 310 may also include an adhesive to adhere to the body of the user.

The top sheet is made of materials which are compliant and readily conform to the shape of the body. The top sheet may exhibit good strikethrough and rewet characteristics, permitting bodily discharges to rapidly penetrate therethrough to the core, but not flow back to the skin of the wearer. The top sheet may be porous to permit discharged liquids to pass through to the core. The body contacting surface provides comfortable and dry-feeling contact with body surfaces while allowing free passage of fluids therethrough into the absorbent core.

The backing sheet 328 prevents discharges absorbed by the core, from escaping. The backing sheet 328 prevents the fluids which are expelled, or which escape from the absorbent core from soiling the user's garments.

The absorbent core is intermediate the top sheet 330 and the backing sheet 328 and the article 310 is integrally or unitarily assembled. The absorbent core has two opposed faces, one oriented towards the backing sheet 328 and one oriented towards the top sheet 330. The absorbent core provides for receiving, collecting and containing bodily discharges such as menses, deposited thereon or which pass through the top sheet 330. In an embodiment, the core is configured to be narrow and thin so as to be comfortable to the wearer.

The wing elements 332i and 332ii extend outwardly from the margin or border 340 of the main part 342 of the auxiliary body 314 and define respective top surfaces 344 and respective bottom surfaces 346 thereof. The bottom surfaces 344 may include adhesive material. The wing elements 332i and 332ii may be provided in a variety of suitable shapes and when in use extend beyond the undergarment crotch portion to be flipped thereunder and adhered to the outer surface of the undergarment opposite the body contacting surface thereof.

The article 310 comprises such as lateral portions such as a pair of wings or flaps 322A and 322B which extend beyond each margin 320A and 120B. Each later portion 322A and 122B respectively and define a respective outer free edge 323A and 323B.

The auxiliary body 314 can be provided in a variety of sizes and shapes thereby extending about the main body 312 with greater or lesser surface area.

In an embodiment, the material used for the auxiliary body 314 is similar to that of the protective layer (sheet 338) of the pad. In an embodiment, the additional material for auxiliary body 314 is hot-pressed or glued to the main body 312 so that when detached therefrom it will not affect the integrity of the main body 312. The glue surface can be thicker or thinner depending on the size of the pad and the nature of the pad. Some pads require more wing support than others thus the variation. The hot press pattern and or glue will vary according to the required force. In an embodiment, the longitudinal ends 334′ and 334″ of the auxiliary body 314 are not attached to the ends 336′ and 336″ of the main body 312 allowing the tearing from the front to back in the case of no wing requirement. In an embodiment, and as shown in FIG. 19, the ends 334′ and 334″ include tear lines 345 allowing for peeling off the auxiliary body 314 from front to back even when the body 314 is in full adhesive contact with the main body 312.

Generally stated and in accordance with an embodiment, there is provided a sanitary article comprising a main body defining opposite longitudinal ends thereof, opposite lateral sides thereof and comprising a top side, an opposite bottom side and an absorbent core therebetween bounded by a core border formed in the top and bottom sides. The top side defines a body contacting surface within the core border. The bottom side defines a garment contacting surface within the core border. An inner contour extends beyond the core border and is bounded by a sealed inner contour border. An outer contour extends beyond the inner contour border and is bounded by a perimeter. The outer contour defines lateral outer contour portions at respective ones of the opposite lateral sides. A pair of wings respectively comprise an inner lateral portion extending beyond a respective one of the lateral outer contour portions and comprises an inner lateral portion being contiguous with the respective one of the lateral outer contour portions and forming a respective longitudinal junction therewith comprising tear line along a length thereof. The longitudinal junction comprises compressed material providing for being torn about the tear line. Each wing is selectively removable from the sanitary article about the tear line.

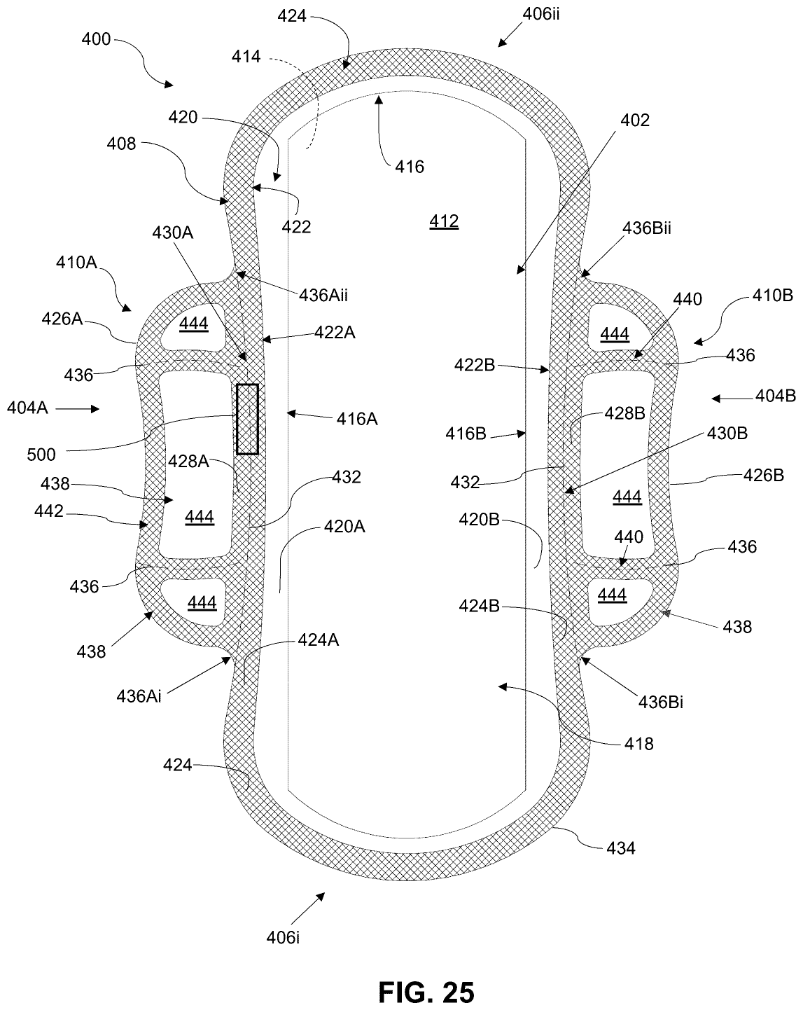

With reference to FIG. 25, a sanitary article 400 in accordance with an illustrative embodiment of the present disclosure is herein described.

In an embodiment, sanitary article 400 provides improvements to the sanitary article disclosed in International Application number PCT/CA2019/000174 filed on 30 Dec. 2019 which is incorporated herein by reference in its entirety.

Article 400 defines a top (body contacting) side 402, an opposite rear (garment contacting) side (not shown), opposite lateral sides 404A and 404B and opposite front and rear ends 406i and 406ii. Article 400 comprises a main central body 408 and lateral portions or wings 410A and 410B extending from the main body 408 at each lateral side 404A and 404B thereof respectively.

The main body 408 is a laminated structure comprising a top sheet 412 that overlies an absorbent core 414 which is sandwiched between the top sheet 412 and the backing sheet (now shown but previously described herein). The absorbent core 414 is bounded by a core border 416 formed with the top sheet 412 and the backing sheet. The area within the border 416 is the core area 418. The top sheet 412 and backing sheet extend beyond the border 416 providing an inner contour portion 420 surrounding the core border 416 and being bounded by an inner contour border 422. The core border 416 defines lateral margins or border lines 416A and 416B at each lateral side 404A and 404B, respectively. The inner contour border 422 defines lateral margins or border lines 422A and 422B at each lateral side 404A and 404B, respectively. The main body 408 comprises an outer contour portion 424 extending beyond the inner contour border 422. The outer contour portion 424 is made of a more dense and/or compressed material which can be provided by heat seal embossing or stamping. Indeed, the density of the outer contour portion 424 is greater than that of the inner contour portion 420 and the core area 418. The outer contour portion 424 defines lateral outer portions 424A and 424B at each lateral side 404A and 404B, respectively.

The wings 410A and 410B are respectively contiguous with the lateral outer portions 424A and 424B. Each wing 410A and 410B defines a respective outer free edge 426A and 426B and comprises a respective inner lateral portion 428A and 428B. Wing lateral portion 428A contiguously extends from lateral outer contour portion 424A forming a junction 430A therebetween and comprising the same dense/compressed material therewith. Wing lateral portion 428B contiguously extends from lateral outer contour portion 424B forming a junction 430B therebetween and comprising the same dense/compressed material therewith. Junctions 430A and 430B comprise respective tear lines 432.

The main body 208 defines an outer perimeter 434 that merges with the free edge 426A at front and rear junctions 436Ai and 436Aii at lateral side 404A and that merges with the free edge 426B at front and rear junctions 436Bi and 436Bii at lateral side 404B.

The wings 410A and 410B can be removed from the main body 408 by tearing along the tear line 432 starting at one of the junctions 436Ai or 436Aii (for wing 410A) and 436Bi or 436Bii (for wing 410B). The junctions 430A and 430B provide sufficient material width to allow enough material for pulling the wings 410A and 410B away from the main body 408 about the tear lines 432. Indeed, this material width is provided by the width of the outer contour portions 424A, 424B and their adjacent wing lateral portion 428A, 428B, respectively contiguously extending therefrom.

The dense material at the junctions 430A and 430B protects the integrity of the lateral inner contour border lines 422A and 422B and thus the integrity of the main body 408 after removal of the wings 410A and 410B. The junctions 430A, 430B are made of hardened, dense material that provides for a more sharp or clean tear along the tear line 432 reducing the probability of an unwanted tear within the outer contour portions 424A an 424B towards the lateral inner contour border lines 422A and 422B. Thus, the integrity of the inner contour portion 420 is protected. Moreover, inner contour portion 420 acts as a spacer between the lateral inner contour border lines 422A and 422B which are contiguous with the junctions 430A and 430B at the core border 416 thereby further protecting the core area 418. Similarly, the outer contour portions 424A, 424B act as spacers between the tear line 432 and the inner contour border lines 422A and 422B. The foregoing configuration provides for maintaining the integrity of the main body 408 during tearing off of the wings 410A and 410B.

Once the wings 410A and 410B are removed the main body 408 provides a standalone sanitary article with the cut tear line 432 now forming part of the perimeter 434.

Each wing 410A and 410B includes inner tear lines 436 extending from their free edges 426A and 426B to the lateral tear line 432 dividing wings 426A and 226B into respective wing sub-parts 438. The wings 426A and 426B comprise respective innerjunction portions 440 containing the inner tear line 436. As such, the junctions 440 are positioned between and contiguous with adjacent wing sub-parts. The junctions 440 extend from the free edges 426A, 426B and merge with the wing lateral portions 428A, 428B. The junctions 440 are made of the same material as the wing lateral portions 428A, 428B. The wings 410A and 410B comprise respective outer contour portions 442 that merge with the lateral portions 428A, 428B and with the inner junctions 440 providing islands 444 therebetween of non-hardened, non-compressed and less dense material similar to that of contour 420.

The junction areas containing the tear lines are heat sealed by heat seal embossing or heat seal stamp which creates a stronger, more compressed and dense material.

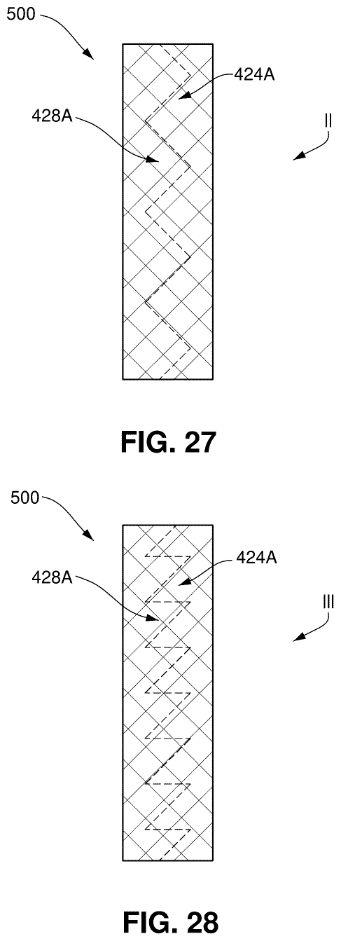

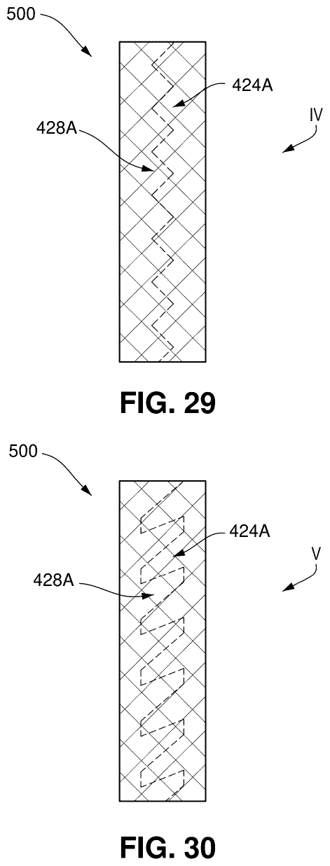

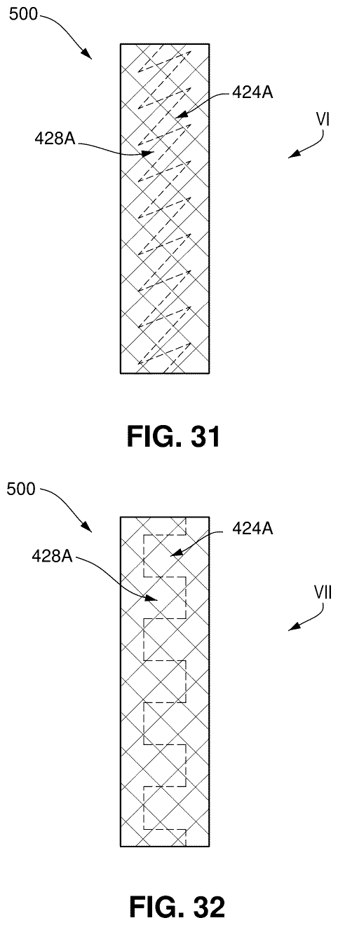

The tear line 432 is generally straight (linear) yet other types of interdigitating tear lines are provided as shown in FIGS. 24-32.

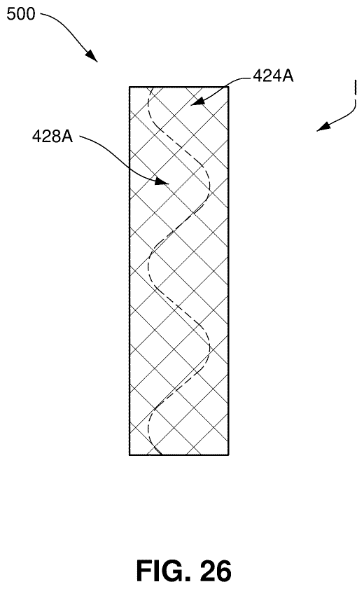

In FIG. 25, a section 500 of the junction 430A is shown containing the tear line 432. FIGS. 24-32 show section 500 of the junction 430A with various tear line patterns that allow for the material on either side of the tear line to interdigitate. More specifically, the lateral outer contour portion 424A interdigitates with the wing inner lateral portion 228A about the tear lines patterns I, II, III, IV, V, VI and VII respectively shown in FIGS. 26, 27, 28, 29, 30 31, and 32.

There is thus provided an interdigitating pattern of the opposite materials about a tear line that runs along a length of the junction 430 in a side to side direction flow about a width of the junction area 56 thereby being reciprocally directed from the wing side to the main body side and then back again in sequential repetition along this length.

The foregoing patterns provides greater stability about the tear line, requiring greater force when removing a wing 410A, 410B from the main body 408 and as such, avoid wings from being accidentally removed from the main body.

The lateral pulling forces during tearing are not perfectly horizontal, they could be upwardly or downwardly directed, usually diagonally with a greater or lesser inclination, relative to a center line along the junction comprising the tear line. As such, the cuts are formed not along a single line pathway but in a side to side pathway (providing the interdigitating configuration described above). The tear line pathway is designed in such a way as to modulate the tearing force required to separate the two materials about the tear line along the length thereof. Indeed, the weakness caused by the tear line is dampened by its non-linear path thereby offsetting weak areas to avoid unwanted or accidental tearing.

Basically, the pulling force of the wing is different in different areas along the cutting line and thus more critical in different spots. In order to assure the full integrity of the wing when in use, the design of the pathway of the cutting line allows for more strength in critical places. The pulling force between pad and wing is not the same and thus critical spots between the pad and the wing are identified and the cut line is designed accordingly to allows for maximum tensile strength at the weakest spots.

The ultimate tensile strength is the point at which a material fails. Strength is a measure of a material's ability to resist an applied load. However, the material will first deform elastically and then plastically before finally breaking into two or more pieces. The ultimate tensile strength is the force the material withstands before it breaks into two or more pieces. The ultimate tensile strength is also known as tensile strength. Sometimes ultimate tensile strength can be written as the acronym “UTS” or “TS”. The rate of elongation and load of the force applied can be used to calculate the material properties. The ultimate tensile strength (UTS) of a material can be found by dividing the force required to break a material by its original cross-sectional area. Strength (UTS)=Force/Area. Force=UTS×Area. Usually, the greater the area, the greater the force required. When applying small cuts between the pad and the wing we reduce the area a thus the force required thereby allowing the possibility of tearing the wings while applying a local force at the upper part of the wing.

Different cut sizes in along the length of the cut line are provided in accordance with the identified critical spots which require more material relative to cuts (i.e. smaller relative cuts) thus providing necessary strength of material.

Where more resistance (against a tearing force) is required, the cuts will be smaller and where we less resistance is required (against a tearing force), the cuts will be bigger. Length of uncut vs cut portion along the seal line provides for modulating the tearing force required.

The foregoing prevents the wing from being accidentally separated from the pad.

In an embodiment, the junction areas between the wing and pad body are made of a more dense compressed material facilitating cutting. In an embodiment, the junction area is heat sealed by heat seal embossing or heat seal stamp which creates a stronger, more compressed and dense material.

Nonwovens consist of individual fibers or endless filaments (fibers of unlimited length) which form a loose cohesion. Nonwovens with thermoplastic parts (plastics which are shaped when heat is applied) can be welded using ultrasonics. Here, the plastic part of the material is heated by ultrasonics, melted and the nonwovens can be connected together (welded) without adhesive. oining nonwovens without additives is perfect for hygiene products.

By providing a more dense and compressed material at the junction, the junction material is made thinner allowing for a “cleaner” cut but with greater tensile strength required in achieving such a cut. The wing is “sliced” off the main body with clean or straight wing and main body edges after separation rather than having jagged edges with protruding filaments and cracks.

In an embodiment, the whole wing is compressed in a similar way (via heat seal for example) and in this way, when the wing includes inner cuts, the sub-parts are “sliced” off in clean manner without jagged edges and protruding filaments.

The skilled artisan will readily appreciate that tear lines include without limitation weak lines, perforated lines which may or may not include dotted lines for allowing the user to tear the portions at each side of the tear line along the dotted line as is known in the art.

Generally stated and in accordance with an embodiment, there is provided sanitary article comprising a main body a pair of wings.

The main body comprises a top side, an opposite bottom side and an absorbent core therebetween surrounded and bounded by a core border formed in the top and bottom sides. The top side defines a body contacting surface within the core border. The bottom side defines an undergarment contacting surface within the core border. A main body contour extends beyond the core border and is surrounded and bounded by a sealed main body perimeter defining opposite main body longitudinal ends and opposite main body lateral sides of the main body. Each of the main body lateral sides having front and rear portions thereof and a middle portion therebetween.

Each of the pair of wings laterally extend from the middle portion of a respective one of the main body lateral sides. Each of the pair of wings define an inner lateral part being contiguous with the middle portion of a respective one of the main body lateral sides and an outer free edge defining front and rear edge sections thereof interconnected by a middle edge section therebetween. The front and rear edge sections laterally extend away from the middle portion of a respective one of the main body lateral sides and the middle edge section is laterally spaced apart from the middle portion of a respective one of the lateral sides. Each of the pair of wings have a respective undergarment contacting portion having an adhesive for adhesively contacting the undergarment of a wearer. When adhesively contacting the undergarment of the wearer, each of the pair of wings extends beyond a crotch portion of the undergarment and is flipped thereunder and adhered to an outer surface of the undergarment opposite a body contacting surface of the undergarment.

Each of the pair of wings defines with the main body a respective front corner and a respective rear corner. The front corner is formed at a junction between the front edge section of a respective one of the pair of wings and the front lateral side portion of a respective one of the main body lateral sides. The rear corner is formed at a junction between the rear edge section a respective one of the pair of wings and the rear lateral side portion of a respective one of the main body lateral sides.

A respective tear line is defined between the inner lateral part of each one of the pair of wings and the middle portion of a respective one of the main body lateral sides in contiguity therewith and extends from the front corner to the rear corner along a length of the inner lateral part of each one of the pair of wings and the middle portion of a respective one of the main body lateral sides in contiguity therewith. Each of the respective tear lines provides for both maintaining each of the pair of wings attached to the middle portion of a respective one of the main body lateral sides when each of the pair of wings is adhesively contacting the outer surface of the undergarment and for detaching each of the pair of wings from the middle portion of a respective one of the main body lateral sides in contiguity therewith when a sufficient tearing force is applied thereto thereby providing for fully removing each of the pair of wings from the main body without physical integrity disturbance of the sealed perimeter at each of the middle portions.

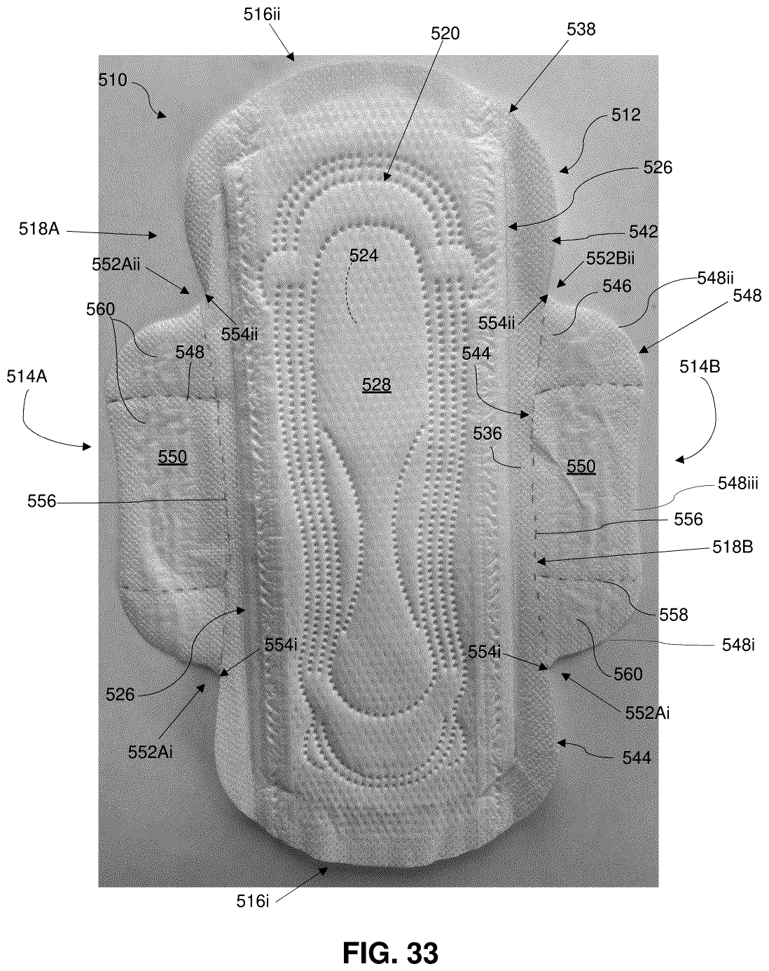

With reference to FIGS. 33 to 36, a sanitary article 510 in accordance with an illustrative embodiment of the present disclosure is herein described.

In an embodiment, sanitary article 510 provides improvements to the sanitary article disclosed in International Application number PCT/CA2019/000174 filed on 30 Dec. 2019 which is incorporated herein by reference in its entirety.

The sanitary article 510 comprises a longitudinal main body 512 with a pair of wings 514A and 514B laterally extending therefrom.

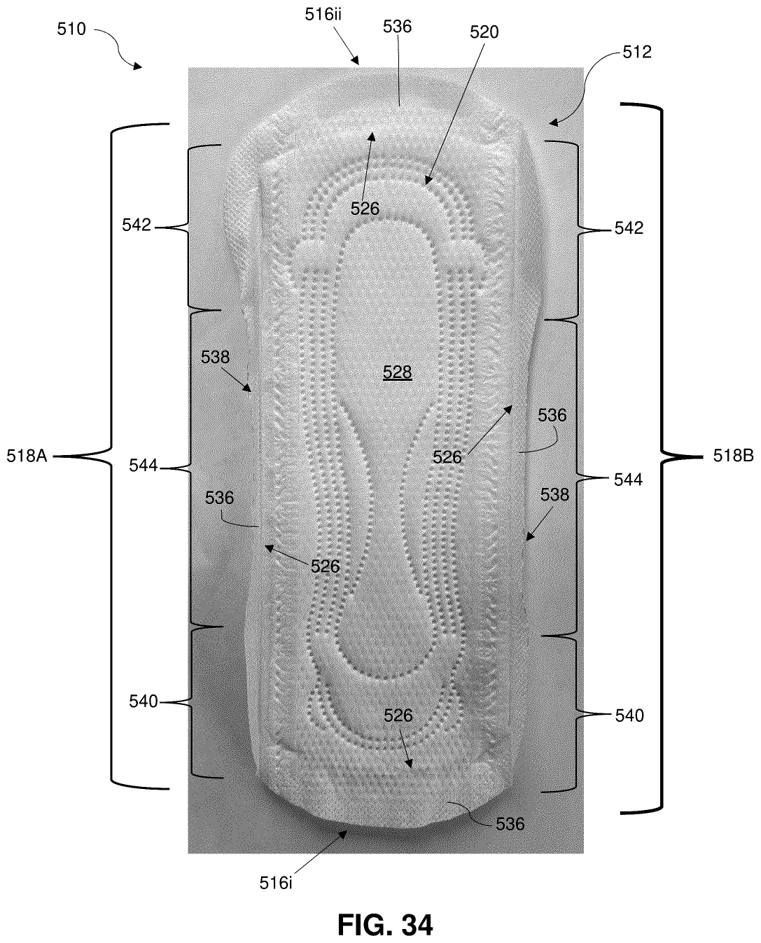

The main body 512 defines opposite longitudinal ends 516i and 516ii and opposite lateral sides 518A and 518B respectively including the removable wings 514A and 514B. In this example, the end 516i is a front end, end 516ii is a rear end, lateral side 518A is a right side and lateral side 518B is a left side. The foregoing depends on the direction of the article when in use. As such, the front and rear ends are reversible, and consequently the right and left sides are likewise reversible depending on the direction of article 510 when in use. Therefore, the terms “front” and “rear” and “left” and “right” are used hereinbelow as indicative of one of the two given reversible directions. It should also be noted that the wings 514A and 514B are removable as shown in FIGS. 34-36 and as such, the lateral sides 518A and 518B become the lateral sides of the article 510 in this case.

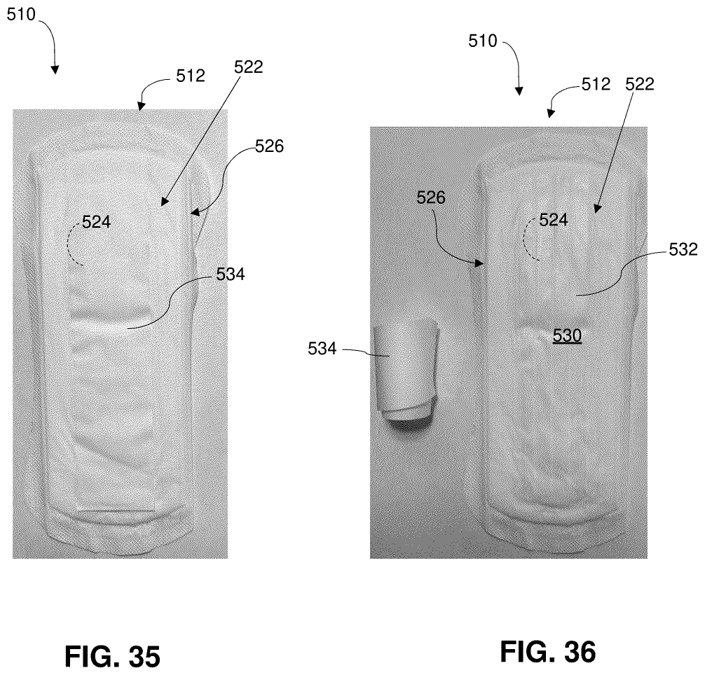

The main body 510 comprises a top side 520 (shown in FIGS. 33 and 34) and an opposite bottom side 522 (shown in FIGS. 35 and 36). An absorbent core 524 is interposed between the top side 520 and the bottom side 522 and is surrounded and bounded by a core border 526 formed in the top 520 and bottom 522 sides. The core border 526 is sealed thereby sealing the absorbent core within the space provided between the top and bottom sides 520, 522 and the core border 526.

The top side 522 defines a body contacting surface 528 within the core border 526 and provides for receiving discharge. The bottom side 524 defines an undergarment contacting surface 530 within the core border 526. As shown in FIGS. 35 and 36, the undergarment contacting surface 530 includes an adhesive material 532 covered by a paper film 534 when not in use for being adhesively connected to the inner part of the crotch of an undergarment.

The main body 510 comprises main body contour 536 that extends beyond the core border 526 and is surrounded and bounded by a sealed main body perimeter 538 defining the opposite longitudinal ends 516i and 516ii and opposite lateral sides 518A and 518B. In an embodiment, the contour 536 is a laminated material formed by the top and bottom sides 520 and 522 when heat sealed together.

Each of the main body lateral sides 518A and 518B define front and rear portions 540 and 542 respectively and a middle portion 544 therebetween.

Each wing 514A and 514B laterally extends from the middle portion 544 of a respective lateral side 518A and 518B. Each wing 514A and 514B defines a respective inner lateral part 546 being contiguous with the middle portion 544 of a of a respective lateral side 518A and 518B. Each wing 514A and 514B has an outer free edge 548 defining front and rear edge sections 548i and 548ii interconnected by a middle edge section 548iii therebetween. The front and rear edge sections 548i, 548ii laterally extend away from the middle portion 544 of a respective lateral side 518A and 518B and the middle edge section 548iii is laterally spaced apart from the middle portion 544 of a respective lateral side 518A and 518B.

Each wing 514A and 514B has a top side 550 and an opposite bottom side (not shown) with an undergarment contacting portion having an adhesive for adhesively contacting the outer side of the crotch portion of an undergarment of a wearer. When adhesively contacting the undergarment of the wearer, each of the wings 514A and 514B extends beyond the crotch portion of the undergarment and is flipped thereunder and adhered to the outer surface of the undergarment which is opposite the body contacting surface of the undergarment on which the main body 510 is positioned.

The wings 514A and 514B respectively define with the main body 510 a pair of laterally opposite front corners 552Ai and 552Bi and a pair of laterally opposite rear corners 552Aii and 552Bii. Each front corner 552Ai and 552Bi is formed at a junction 554i between the front edge section 518i of each wing 514A and 518B and the front lateral side portion 540 of a respective lateral side 514A and 518B. Each rear corner 552Aii and 552Bii is formed at a junction 554ii between the rear edge section 518ii of each wing 514A and 514B and the rear lateral side portion 542 of a respective lateral side 518A and 518B.

A respective tear line 556 is defined between the inner lateral part 546 of each wing 514A and 514B and the middle portion 544 of a respective a respective lateral side 518A and 518B in contiguity therewith and extends from the the front corners 552Ai and 552Bi to the rear corner 552Aii and 552Bii along a length of the inner lateral part 546 of each wing 514A and 514B and the middle portion 544 in contiguity therewith. The tear line 556 is stippled with each strand of connected material followed by a strand of perforated cuts

Each of these tear lines 556 provides for maintaining each wing 514A and 514B attached to the middle portion 544 in contiguity therewith when each of the pair of wings is adhesively contacting the outer surface of the crotch portion of the undergarment. As such, the tear lines 556 are configured to maintain connection and not be easily broken. The tear lines 556 also provide for detaching each wing 514A and 514B from the middle portion in contiguity therewith when a sufficient tearing force is applied thereto thereby providing for fully removing the wings 514A and 514B from the main body 512 without physical integrity disturbance of the sealed perimeter 538 at each of the middle portions 544.

As such, the main perimeter 548 of the main body 510 is not damaged during tearing as sufficient threshold tearing force is required to detach the wings 514A and 514B from the main body 510 otherwise, without such strong connection between the middle portions 544 and the wings 514A and 514B, the wings 514A and 514B may be easily removed especially when in use as the wearer moves. In an embodiment, this additional connective force between the main portions 544 of the wings 514A, 514B is accomplished by providing a greater amount of connected material along the tear line 556 and a lesser amount of open cuts. The front corners 552Ai and 552Bi and rear corners 552Aii and 552Bii provide a suitable starting place for initiating tearing along the tear line 556 as they provide gripping with one hand the main body and with the other hand the wing about the V-shaped valley-like corner.

In an embodiment, each wing 514A and 514B comprises inner tear lines 558 for separating each wing 514A, 514B into sub-parts 560 thereof.

The various features described herein can be combined in a variety of ways within the context of the present disclosure so as to provide still other embodiments. As such, the embodiments are not mutually exclusive. Elements of the embodiments and examples discussed can be combined within the context of the disclosure as those having skill in the art will readily appreciate. Moreover, the embodiments discussed herein need not include all of the features and elements illustrated and/or described and thus partial combinations of features can also be contemplated. Furthermore, embodiments with less features than those described can also be contemplated.

It is to be understood that the present disclosure is not limited in its application to the details of construction and parts illustrated in the accompanying drawings and described hereinabove. The disclosure is capable of other embodiments and of being practiced in various ways. It is also to be understood that the phraseology or terminology used herein is for the purpose of description and not limitation. Hence, although the present disclosure has been provided hereinabove by way of non-restrictive illustrative embodiments thereof, it can be modified, without departing from the scope, spirit and nature thereof and of the appended claims.

What is claimed is:

1. A sanitary article comprising:
a longitudinal main body comprising a top body contacting surface, an opposite bottom undergarment contacting surface, and an absorbent core therebetween, the top body contacting surface and the bottom opposite outer surface being laminated to define laminated top and bottom surfaces formed about the absorbent core thereby defining an absorbent core sealed border line, the longitudinal main body is delimited and bounded by a perimeter defining sealed opposite ends and sealed opposite lateral margins, the sealed opposite ends define a main body length therebetween, the sealed opposite lateral margins define a main body width thereof and run along the length of the main body main, the lateral margins define respective lateral margin lengths thereof, the sealed opposite margins being respectively spaced apart from the absorbent core sealed border line by the laminated top and bottom surfaces therebetween; and
two lateral wings respectively laterally extending from the main body beyond a respective one of the sealed opposite lateral margins and defining respective outer free edges thereof, each one of the two lateral wings having an undergarment contacting portion having an adhesive for adhesively contacting the undergarment of a wearer, wherein when adhesively contacting the undergarment of the wearer, each one of the two lateral wings extends beyond a crotch portion of the undergarment and is flipped thereunder and adhered to an outer surface of the undergarment opposite a body contacting surface of the undergarment, each one of the two lateral wings comprising a respective tear line formed thereon and being positioned adjacent of and spaced apart from a respective one of sealed opposite lateral margins running along the respective lateral margin length thereof and extending from one part of the free edge to an opposite part of the free edge, the respective tear line of each one of the two lateral wings defining an outer lateral wing portion and an inner lateral wing portion, the inner lateral wing portion being positioned between the tear line and the adjacent sealed lateral margin,
wherein the respective tear line of each one of the pair of lateral wings provides for both maintaining the outer lateral wing portion attached to inner lateral wing portion when each of the lateral wings is adhesively contacting the undergarment and for detaching the outer lateral wing portion from the inner lateral wing portion when a sufficient tearing force is applied thereto thereby providing for fully removing each of the lateral wings from the longitudinal main body without physical integrity disturbance of each of the sealed opposite lateral margins.

2. A sanitary article according to claim 1, wherein:
each of the sealed opposite lateral margins defining front and rear portions thereof and a middle portion therebetween;
each of the two lateral wings respectively laterally extending from the middle portion of a respective one of the sealed opposite lateral margins;
each of the outer free edges of each of the two lateral wings defining front and rear edge sections thereof interconnected by a middle edge section therebetween, the front and rear edge sections laterally extending away from the middle portion of a respective one of the sealed opposite lateral margins and the middle edge section being laterally spaced apart from the middle portion of a respective one of the sealed opposite lateral margins;
each of the two lateral wings defining with the main body a respective front corner and a respective rear corner, the front corner formed at a junction between the front edge section of a respective one of the two lateral wings and the front portion of a respective one of the sealed opposite lateral margins, the rear corner formed at a junction between the rear edge section a respective one of the two lateral wings and the rear portion of a respective one of the sealed opposite lateral margins, and
the tear line extending from the front corner to the rear corner along a length of the middle portion of a respective one of the sealed opposite lateral margins.

3. A sanitary article according to claim 1, wherein the tear line comprises cut lines having relatively longer and short lengths for modulating tensile strength along the tear line.

4. A sanitary article according to claim 1, wherein the tear line provides for an interdigitating pattern between outer lateral contour portion of the main body and the inner lateral portion of an adjacent one of the pair of wings along the tear line.

5. A sanitary article according to claim 1, wherein the tear line of at least one of the lateral wings is a first tear line defining a first lateral margin length thereof and wherein the at least one lateral wing comprises a second tear line formed thereon and being positioned adjacent of and spaced apart from the first tear line running along the first lateral margin length thereof, wherein the second tear line separates the outer portion of the lateral wing into a first outer portion and a second outer portion, wherein the second outer portion is positioned between the first and second tear lines, wherein the second tear line provides for detaching the first outer portion from the second outer portion and wherein the first tear line provides for detaching the second outer portion from the inner portion.

6. A sanitary article according to claim 1, wherein the first and second outer portions comprise respective indicia.

7. A sanitary article according to claim 1, wherein the opposite bottom undergarment contacting surface comprises an adhesive for adhesively connecting to the undergarment.

8. A sanitary article comprising:
a main body comprising a top side, an opposite bottom side and an absorbent core therebetween surrounded and bounded by a core border formed in the top and bottom sides, the top side defining a body contacting surface within the core border, the bottom side defining an undergarment contacting surface within the core border, a main body contour extending beyond the core border and surrounded and bounded by a sealed main body perimeter defining opposite main body longitudinal ends and opposite main body lateral sides, each of the main body lateral sides having front and rear portions thereof and a middle portion therebetween; and
a pair of wings, each of the pair of wings laterally extending from the middle portion of a respective one of the main body lateral sides, each of the pair of wings defining an inner lateral part being contiguous with the middle portion of a respective one of the main body lateral sides and an outer free edge defining front and rear edge sections thereof interconnected by a middle edge section therebetween, the front and rear edge sections laterally extending away from the middle portion of a respective one of the main body lateral sides and the middle edge section being laterally spaced apart from the middle portion of a respective one of the lateral sides, each of the pair of wings having a respective undergarment contacting portion having an adhesive for adhesively contacting the undergarment of a wearer, wherein when adhesively contacting the undergarment of the wearer, each of the pair of wings extends beyond a crotch portion of the undergarment and is flipped thereunder and adhered to an outer surface of the undergarment opposite a body contacting surface of the undergarment,
each of the pair of wings defining with the main body a respective front corner and a respective rear corner, the front corner formed at a junction between the front edge section of a respective one of the pair of wings and the front lateral side portion of a respective one of the main body lateral sides, the rear corner formed at a junction between the rear edge section a respective one of the pair of wings and the rear lateral side portion of a respective one of the main body lateral sides, and
a respective tear line defined between the inner lateral part of each one of the pair of wings and the middle portion of a respective one of the main body lateral sides in contiguity therewith and extending from the front corner to the rear corner along a length of the inner lateral part of each one of the pair of wings and the middle portion of a respective one of the main body lateral sides in contiguity therewith;
wherein each of the respective tear lines provides for both maintaining each of the pair of wings attached to the middle portion of a respective one of the main body lateral sides when each of the pair of wings is adhesively contacting the outer surface of the undergarment and for detaching each of the pair of wings from the middle portion of a respective one of the main body lateral sides in contiguity therewith when a sufficient tearing force is applied thereto thereby providing for fully removing each of the pair of wings from the main body without physical integrity disturbance of the sealed perimeter at each of the middle portions.

9. A sanitary article according to claim 8, wherein the inner lateral portion of each of the pair of wings and the middle portion of a respective one of the main body lateral sides in contiguity therewith comprise compressed material.

10. A sanitary article according to claim 9, wherein the compressed material is heat sealed.

11. A sanitary article according to claim 8, wherein the tear line comprises cut lines having relatively longer and short lengths for modulating tensile strength along the tear line.

12. A sanitary article according to claim 8, wherein the tear line provides for an interdigitating pattern between the inner lateral portion of each of the pair of wings and the middle portion of a respective one of the main body lateral sides in contiguity therewith along the tear line.

13. A sanitary article according to claim 8, wherein the undergarment contacting surface of the main body comprises an adhesive for adhesively connecting to the undergarment.

14. A sanitary article according to claim 8, wherein each of the pair of wings comprises inner tear lines for separating each of the pair of wings into sub-parts thereof.
